(19) United States
(12) Patent Application Publication
(10) Pub. No.: US 2018/0243446 A1
(43) Pub. Date: Aug. 30, 2018

(54) METHOD AND COMPOSITIONS FOR REMOVING DUPLICATED COPY NUMBER VARIAIONS (CNVS) FOR GENETIC DISORDERS AND RELATED USES

(71) Applicant: THE HOSPITAL FOR SICK CHILDREN, Toronto (CA)

(72) Inventors: Ronald Cohn, Toronto (CA); Daria Wojtal, Toronto (CA); Dwi Kemaladewi, Toronto (CA); Zhenya Ivakine, Burlington (CA)

(73) Assignee: The Hospital for Sick Children, Toronto (CA)

(21) Appl. No.: 15/756,837

(22) PCT Filed: Sep. 1, 2016
(86) PCT No.: PCT/CA2016/051041

§ 371 Date: Mar. 1, 2018

Related U.S. Application Data
(60) Provisional application No. 62/212,934, filed on Sep. 1, 2015.

(51) Int. Cl. A61K 48/00; A01K 67/027; A61K 38/46; C12N 15/113; C12N 15/90; C12N 9/22

(52) U.S. Cl. CPC A61K 48/0066; A01K 67/0275; A61K 38/46; A61K 48/0016; C12N 15/113; A61P 21/00; C12N 9/22; A01K 2217/072; A01K 2227/105; A01K 2267/0306; C12N 15/907

(57) ABSTRACT

In one aspect, the Invention relates to methods and compositions to remove replicate genetic material, such as duplicate copy number variations (CNVs) present in genetic material using targeted endonuclease technology with one guide, such as one single guide RNA. In another aspect, the invention relates to methods and compositions useful for removing replicate or duplicate genetic material. In another aspect the invention is useful for the treatment and prevention of conditions that are caused by replicate genetic material, including duplicate CNVs, such as a certain types of Duchenne Muscular Dystrophy and or MECP2 duplication syndrome. In another aspect the invention provides novel genetically engineered animals with duplicate CNVs useful to screen potential therapies.

### CROSS REFERENCE

This application claims priority from United States Provisional Patent Application, U.S. 62/212/934, filed Sep. 1, 2015, the entirety of which is incorporated herein by reference.

### FIELD OF THE INVENTION

In one aspect, the invention relates to methods and compositions for removing replicated genetic material, such as duplicated copy number variations (CNVs) and related uses. In another aspect, the invention relates to methods and compositions for the treatment or prevention of conditions relating to replicate genetic material such as duplicate CNVs, including but not limited to certain Duchenne Muscular Dystrophies and MECP2 duplication syndrome. In another aspect, the invention provides genetically engineered animals having duplicate CNVs and methods for producing same.

### INCORPORATION BY REFERENCE STATEMENT

All references and sequence listing as noted herein are incorporated by reference. The paper copy and computer-readable form (CRF) are identical. The Sequence Listing forms part of this application.

### BACKGROUND OF THE INVENTION

The use of endonucleases as targeted gene editing tools has rapidly emerged with the advent of technologies such as the use of zinc finger proteins, TALENS and CRISPR/Cas9 systems. Zinc fingers are dimers comprising Zinc fingers which recognize DNA basepair in triplets and the Fokl nuclease, cutting in the spacer region between two distinct ZF target sites. A TAL effector nuclease (TALEN) is similar in principle to the ZF nuclease, comprising the components of the array which recognize individual nucleotides and the Fokl nuclease cutting in the spacer region between two distinct TALENS target sites.

Clustered regularly interspaced short palindromic repeats (CRISPR)-Cas systems are adaptive immune systems used by many bacteria and archaea to fight off foreign DNA in the form of bacterial phages and/or plasmids (1). Specifically, the type II CRISPR/Cas system works through RNA-directed endonuclease cleavage of the invading genomic sequence. The invading sequence is captured and inserted directly into the genome of the host organism between CRISPR regions (2-4). Following transcription and processing of these loci, RNA guided endonucleases are made with the capability to target foreign nucleic acids based on complementarity with the RNA (5).

Since realizing the potential power of a programmable nuclease in editing mammalian genomes, the CRISPR/Cas9 system has been developed as a technology for multiple biological contexts (6, 7). Regardless of the platform, this system requires a mammalian optimized Cas9 protein and a chimeric single guide RNA (sgRNA) which is made up of CRISPR RNAs (crRNA) and a trans-activating CRISPR RNA (tracrRNA) (6-9). The guide sequences are generally 17-20-bp long (10). Target sequences must be adjacent to a protospacer adjacent motif (PAM) sequence for Streptococcus pyogenes Cas9 (SpCas9) in the form of 5′-NGG (11). Cas9 target recognition is dictated by the Watson-Crick base-pairing of an RNA guide with its DNA target (2, 3). Once expressed in cells, the Cas9 nuclease and the sgRNA form a complex, bind to the target sequence, and make a double-stranded break in the target. The break is repaired via the cellular process of non-homologous end joining (NHEJ), which may introduce insertions and deletions (indels) into the target sequence. Targeted mutations can also be introduced by co-transfecting single- or double-stranded DNA templates to promote homology directed repair (HDR) (See FIG. 1A). To date, the SpCas9 has been used broadly to achieve efficient genome editing in a variety of species and cell types, including human cell lines, bacteria, zebrafish, yeast, mouse, fruit fly, roundworm, rat, common crops, pig, and monkey (reviewed in (12)).

Another application of the CRISPR/Cas9 tool is to regulate gene expression. This approach uses a catalytically inactive or “dead” (dCas9), which when bound to DNA elements may repress transcription by sterically hindering the RNA polymerase machinery (13), likely by stalling transcriptional elongation (See FIG. 1B). Alternative strategies have been developed such as the conversion of Cas9 into a synthetic transcriptional activator by fusing it to multiple copies of VP16 activator (14-16) (See FIG. 1C) Studies from several groups suggest that targeting Cas9 activators using a single sgRNA to a particular endogenous gene promoter leads to only modest transcriptional upregulation (15-17). Instead targeting a promoter with multiple sgRNAs may be a better alternative to increase activation due to synergistic effects (15-17). For instance, using multiple guides, directed to more than on target site, whether it is multiple Zinc finger proteins, or TALEN arrays or sgRNAs.

Gene editing tools such as the site directed endonuclease technology has promise in a number of therapeutic applications in genetic disorders.

One type of genetic disorders that it has not been easily applied are conditions related to duplications of genetic material. With the development of powerful genome analysis platforms, there is growing evidence for the prevalence of copy number variations (CNVs) that are associated with numerous genetic conditions (26). Such conditions include a certain percentage of patients with Duchenne's muscular dystrophy (DMD) and MECP2 duplication syndrome.

However, to date no successful therapeutic strategies have been developed to target these large genomic rearrangements or duplication of genetic material disorders.

### SUMMARY OF THE INVENTION

The present inventors have developed a method of using an engineered targeted endonuclease technology (such as CRISPR/Cas9 technology) that can be used to remove replicate, such as duplicate genetic material using one guide. In some embodiments the invention is useful to modulate expression of genes that are known to play a critical role in disease pathogenesis to therapeutically target autosomal dominant, heterozygous, gain-of-function mutations and large chromosomal rearrangements such as duplications of genetic material. In one aspect, the invention provides a method for specifically targeting duplicate genetic material, such as CNVs, and restoring proper wild type gene expression using one guide, such as one single guide RNA, that targets the replicated (such as duplicated) genetic material.

In one aspect the invention provides a method of removing replicate (such as duplicate) genetic material in vivo, ex vivo or in vitro that is present head to tail on a nucleotide sequence in a eukaryotic cell. In some embodiments the replicate material may or may not have intervening sequences between them. The method in some embodiments comprises using one guide, such as one single guide RNA, and an endonuclease, such as a Cas protein bearing effector domains to cut genetic material, such as DNA at a desired location, in vivo, ex vivo or in vitro in eukaryotic cells. In one aspect, the guide, such as the guide RNA has a region that is coupled to or complexed with the endonuclease, such as a Cas protein and a region that can bind to the target DNA, delivering the endonuclease to the target site. In some embodiments, the guide can be a Zinc finger or TALENS array coupled to or complexed to an endonuclease, such as Fok1.

In some embodiments, the method further comprises a repair or joining of the cleaved genetic material, for instance by non-homologous end-joining or homology directed repair or other suitable repair mechanisms. However, an endonuclease that creates a double stranded break and repaired by non-homologous end-joining is preferred for certain uses of the invention, such as in removing the replicate genetic material and restoring a single copy of the genetic material. In some embodiments, the method can be used to treat or prevent conditions associated with replicate (or duplicate) genetic material, such as certain types of Duchenne's muscular dystrophy (DMD), to for instance restore full-length dystrophin and a-dystroglycan expression or in the treatment of MECP2 duplication syndrome to remove large genome rearrangements.

As such, in one aspect the invention provides a method for treatment of a condition in a mammal that can benefit from deletion of replcated region of genetic material, the method comprising administering to said mammal an effective amount of:
(i) a nucleic acid encoding one guide (which includes one or more copies of the same guide to generate one or more copies of the guide), such as guide RNA, a zinc finger or a TALENS array, complementary (including sufficiently complementary) or binding to a region of genomic DNA encoding the replicate or duplicate region of genetic material of the eukaryotic cell of said mammal (e.g. the genomic target) and a region that can interact and complex with an endonuclease, such as a Cas protein or Fok1, or a composition comprising same, and
(ii) a nucleic acid encoding an endonuclease, such as a Cas protein or Fok1 that interacts and can complex with the guide, such as the guide RNA, Zinc finger or TALENS array and the genomic target or a composition comprising same,

wherein the nucleic acids of (i) and (ii) are incorporated into a vector and in a delivery vehicle suitable for delivering same to the eukaryotic cell of the mammal in a manner that enables the cell to be transfected with said nucleic acids and express same once transfected to remove the duplicate genetic material and restore wild type gene expression. In one embodiment (i) and (ii) are incorporated into the same vector. In another embodiment they are in different vectors. In one embodiment (i) and (ii) are in the same or different compositions. In one embodiment the endonuclease is exogenous to the cell. In another embodiment it is endogenous (.e.g. a transgene) and expressed in the cell or a suitable stimulus is applied to the cell to induce expression of the desired endonuclease.

In another aspect, the invention provides a composition comprising:

(a) one or more of the following:
a nucleic acid encoding a guide, such as a single guide RNA of the invention (wherein in one embodiment the composition comprises one or multiple copies of the nucleic acid encoding the guide, such as a single guide RNA);
(ii) a nucleic acid encoding an endonuclease, such as a Cas protein (such as Cas9) or a Cas fusion protein;
(iii) an endonuclease such as a Cas protein (such as Cas9) or a Cas fusion protein;
(iv) a guide, such as a guide RNA of the invention;
(v) an endonuclease Cas or Cas fusion protein of the invention complexed with the guide RNA of the invention;
(vi) both (iii) and (iv);
(vii) a vector comprising (i) and (ii) or two vectors comprising (i) and (ii) respectively and
(viii) any one or more of the above and optionally a delivery vehicle, such as a bacterial (e.g. plasmid), viral (e.g. phage), cosmid, or other delivery vehicle (for instance in the case of delivery of (iii)-(iv) could be packaged in a liposome); and optionally

(b) a pharmaceutically acceptable carrier.

In another aspect, the invention comprises a kit comprising a composition of the invention and optionally instructions for use.

In other aspects, the invention provides a method of making a genetically engineered animal using an engineered targeted endonuclease technology, such as a CRISPR/Cas9 technology. In one embodiment the genetically engineered animal comprises duplicate genetic material, such as a duplicate CNV, which in some embodiments can be used as an animal disease model for conditions associated with said duplicate CNV. In one embodiment the invention provides a mouse carrying Dmd exons 18-30 duplication and methods and uses thereof. In another embodiment the invention provides vectors comprising the guide and endonuclease, and the guide and endonuclease for making the engineered mouse. Although the invention is not limited to genetically engineered mice the same methodology can be used in other animals, such as rats, hamsters or other animals.

Additional aspects and advantages of the present invention will be apparent in view of the description which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The invention will now be described in relation to the drawings, in which:

FIG. 1. Application of CRISPR. (A) A schematic diagram of a Cas9-gRNA complex interacting with DNA to create either a double strand break (I) or a single-strand nick (II). Double-strand breaks are typically repaired by a non-homologous end joining (NHEJ) process which can introduce nucleotide insertions and deletions (indels) of various lengths in proximity to the cleavage site. Single-stranded nicks are preferentially repaired by homology-directed repair (HDR) in the presence of a donor construct (also referred to as a “repair template”) having homology to the nicked region, resulting in the incorporation of the donor construct nucleotide sequence at the locus. (B) A schematic diagramillustrating use of dCas9 to repress transcription. (C) A schematic diagram illustrating conversion of dCas9 inot a transcriptional activator.

FIG. 2. Targeted deletion of a 143 kp duplication in DMD. (A) Electropherogram of the DMD exons 18-30 duplication junction. An insertion of AAAT at the junction highlighted in blue. (B) Duplication removal strategy using either a single Cas9 guided nuclease specific to a duplicated intron (S6141) or 2 different nucleases: one specific to the junction of the duplication (Junc) and the other to the intron just outside of the duplicated region (S275). (C) PCR strategy using three primers positioned to the duplicated locus. P1 and P3 are universal to the region of interest, whereas P2 is specific to the junction of the duplicated region. (D) PCR of DNA from 3 replicate experiments in which patient myoblasts were transduced with lentiGFP or lentiCRISPR Cas9 nuclease with one guide S6141 or two guides, junc +S275. DNA collected 7 days post puromycin selection. The top band is amplified from universal primers to both an allele with the duplication and single copy. The bottom band is specific to alleles harbouring the duplication. A decrease in the bottom band indicating removal of the duplicated region was only observed in the single guide S6141 strategy. (E) Quantification of band intensities. Graph depicts fold change between top healthy band to bottom duplication band. p=0.05 for S6141 and p=0.314 for junc+ s275. (F) Western blot with anti-dystrophin antibody against Rod terminus and Ponceau stained gel as a loading control. Full-length dystrophin is only detected in dup18-30 myotubes treated with lentiCRISPR+guide S6141 as well as WT myoblasts.

FIG. 3. Genome editing strategies for individual patient mutations in DMD. (A) Electropherogram of the DMD exons 18-30 duplication junction, highlighted in blue is the insertion of AAAT at the junction. (B) Schematic of relative position of Dup18-30: sgRNA 1 to DMD. (C) Schematic of the three-primer duplication removal strategy. P1 and P3 are universal to the region of interest, whereas P2 is specific to the junction of the duplicated region. (D) Targeted deletion of a 139 kb duplication in DMD. PCR of DNA from 3 replicate experiments in which patient myoblasts were transduced with LentiGFP or LentiCRISPR Cas9 nuclease with Dup18-30: sgRNA 1. The top band is amplified with universal primers (P1+P3) to both an allele with the duplication and control. The bottom band is specific to alleles harbouring the duplication (P1+P2). A decrease in the bottom band indicating removal of the duplicated region was only observed when Cas9 and sgRNA 1 were present. (E) Western blot with antibodies against, dystrophin, α-dystroglycan and tubulin as a loading control. Expression level of dystrophin is quantified relative to tubulin by densitometric analysis. *-p<0.05, ** -p<0.01, Student's t-test.

FIG. 4. Targeted deletion of a 114 kb fragment of DNA containing MECP2 gene in WT genomic DNA. (A) Schematic depicting the q28 region (h19) of the X-chromosome which has been duplicated in this patient with MECP2 duplication syndrome. The breakpoints of the duplication, as indicated by the dotted lines, fall within the L1CAM and OPN1LW genes and include intervening genes one of which is MECP2. (B) Relative position of MECP2: sgRNAs 1 and 2 to WT genomic DNA. (C) Presence of targeted deletion assessed using PCR with primers P1 and P2 in human primary fibroblasts nucleofected with corresponding CRISPR components. Intervening fragment between MECP2: sgRNAs 1 and 2 can only be amplified if 114 kb fragment is deleted. Band corresponding to a deletion is only detected in cells nucleofected with the 2 sgRNAs coupled with Cas9. (D) Sequencing read of deletion in human primary fibroblasts with annotated positions of the 2 sgRNAs used.

FIG. 5. Targeted deletion of a 114 kp duplication X-chromosome duplication including MECP2 gene. A single Cas9 guided nuclease (g75 or g80) or no guide GFP control was delivered to patient fibroblasts using lentivirus. DNA was collected at 3 time points, before puromycin selection, 3 days and 7 days post. (A) Gel of a 3 way PCR amplification showing an accumulation of the bottom band corresponding to single copy amplicon and decrease in the top band corresponding duplicated copy. This indicates a removal of the duplicated region. (B) Quantification of band intensity from gel in (A). Graph depicting the ratio between the top band corresponding to duplication in comparison to the bottom band corresponding to healthy single copy. The difference is significant to p=<0.5 at day 3 and p=<0.01 at day 7.

FIG. 6. Targeted removal of a 278 kb X-chromosomal duplication containing the MECP2 gene. (A) Relative position of MECP2: sgRNAs 1 and 2 on the X-chromosome. Target sequences are indicated in highlight in blue and the PAM sequence in highlight in red, being CGG and GGT respectively). (B) Removal of the duplicated region is detected with a PCR strategy using three primers positioned to the duplicated locus. P1 and P3 are universal to the region of interest, whereas P2 will only amplify the duplication junction with P1. (C) A Cas9 nuclease guided by sgRNAs 1, 2, or no sgRNA GFP control was delivered to patient fibroblasts using lentiviral particles. Three-primer PCR demonstrated an accumulation of the bottom band corresponding to the WT single copy amplicon and decrease in the top band corresponding to the duplicated copy. (D) Densitometric analysis of image “c” depicting a decrease in the ratio between the duplicated to the WT band. **-p<0.01, Student's t-test.

FIG. 7 (A) Schematic depicting duplication of exons 18-30 (red) and guides used to generate the dup 18-30 Dmd mouse model. (B) Sequencing read of the duplication junction in mESCs clone 3A7 aligned to a predicted duplication sequence that is based on the prediction that a DSB will occur 3 bp before the PAM of a guide in intron 17 and 30. (C) PCR of tail-tissue using 3 primer PCR strategy that amplifies the duplication junction (Junc) and non duplicated sequence (WT). Of the 5 offspring of one 3A7 chimera, C1-3 F1 females but not C4-5 F1 males, showed germline transmission of this X-linked duplication. The NTC sample is a no DNA template control. (D) Mutation detection assay for three tested sgRNAs targeting intron 21, 26, 27. i26 was the most active sgRNA. The positive control for this mutation detection assay is a PCR from cells transfected with a known active sgRNA. Negative controls are samples where the mutation detection enzyme was not added.

FIG. 8 is a map of plasmid pSpCas9(BB)-2A-Puro (PX459) V 2.0_Guide i17-1 comprising the coding sequence for sgRNA 17-1.

FIG. 9 is a map of plasmid pSpCas9(BB)-2A-Puro (PX459) V 2.0_Guide i17-2 comprising the coding sequence for sgRNA 17-2.

FIG. 10 is a map of plasmid pSpCas9(BB)-2A-Puro (PX459) V 2.0_Guide i30-1 comprising the coding sequence for sgRNA 30-1.

FIG. 11 is a map of plasmid pSpCas9(BB)-2A-Puro (PX459) V 2.0_Guide 130-2 comprising the coding sequence for sgRNA 30-2.

FIG. 12 is a schematic illustrating the method used to make the genetically engineered mouse carrying Dmd exons 18-30 duplication.

FIG. 13 is a map of lentiCRIPR v2_guide_i26 comprising the coding sequence for sgRNA i26.

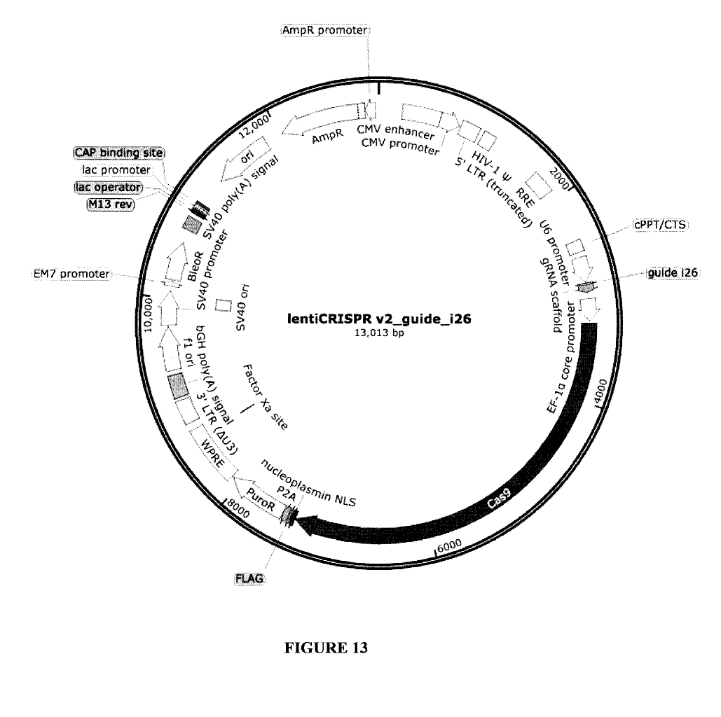

FIG. 14 is a schematic illustrating the process for treating a condition associated with duplicative genetic material using the method of the invention.

FIG. 15 are immunofluorescence stains detecting dystrophin in wildtype mouse (A), the right TA of Dmd exons 18-30 duplication mice after viral injection with sgRNA26i and SpCas into the right TA (B) and (C) illustrating duplication removal in vivo. Panels (D) to (F) are the controls: (D) being the negative control for immunostaining (secondary antibodies) and (E) and (F) being the immunoflurescene stains of the left TA of the mice of (B) and (C).

### DETAILED DESCRIPTION OF THE INVENTION

The present invention provides a method for removing replicated, such as duplicated genetic material, such as genomic material, such as large duplicated genomic rearrangements, using a one guide, such as one single guide RNA, and an endonuclease, such as Cas (Cas9).

In another aspect, the invention provides a method, such as a therapeutic method, for treating conditions related to replicated, such as duplicated, genetic material, such as certain inherited disorders that are caused by duplication of genetic material.

In another aspect, the present inventors have for the first time demonstrated removal of large genomic rearrangements using an engineered targeted endonuclease technology, e.g. CRISPR/Cas9 technology. In one embodiment they have shown it in the X-chromosomal duplication including the MECP2 gene as well as a large duplication of the dystrophin gene in a patient with Duchenne muscular dystrophy. The results demonstrate that large genomic rearrangements caused by copy number variations (CNVs) are amenable to treatment strategies mediated by an engineered endonuclease technology such as the CRISPR/Cas9 methodology.

Using an adapted CRISPR/Cas9 strategy to illustrate their invention, the present inventors have successfully removed a 139 kb duplication (exons 18-30: chrX:32,552,206-32,413,149 (hg19) duplication with an AAAT insertion in the breakpoint junction) in the DMD gene leading to restoration of full-length dystrophin and a-dystroglycan expression in patient myotubes.

In summary, it is herein demonstrated that engineered targeted endonuclease systems, such as CRISPR/Cas9 can have significantly broader therapeutic implications for DMD, which includes strategies to restore full-length dystrophin expression.

An increasing number of genetic disorders are caused by chromosomal rearrangements and CNVs. However, treatments targeting the underlying cause of these disorders are currently not available. Here, the inventors developed a novel strategy employing an engineered targeted endonuclease technology (e.g. the CRISPR/Cas9 system) to remove duplicated regions within the genome. The strategy uses a one guide (e.g., sgRNA) approach, which due to the nature of a tandem (head-to-tail) duplication creates two double stranded breaks. Since the target is a sequence within a duplication, the sgRNA target will be found twice, leading to the formation of two double stranded breaks (“DSB”) and hence removing the intervening sequence which equates to the total size of the duplication. In fact the invention can be used for any replication of sequences not just duplications but also where multiple copies (more than 2) of genetic material are present to leave a single copy of the genetic material, as the guide/endonuclease complex will target each site and remove intervening sequences. Further, although the replications are head to tail, intervening sequences may be present between the replications, which will also be removed upon use of the methods of the invention and the one guide approach presented herein. There are several advantages to this strategy. First, the design of RNA guides is not limited to specific sequences near the breakpoints. This allows for a larger selection of guide RNAs that can target any portion of the duplicated sequence while minimizing off-target sites. Second, given the limited loading capacity of potential in vivo delivery vehicles such as AAV9, strategies using the least amount of CRISPR components will be critical for the development of further therapeutic applications. The inventors have herein successfully removed a large chromosomal rearrangement on the X-chromosome containing the MECP2 gene indicating that this approach can be targeted toward several chromosomal duplication syndromes. Importantly, off-target analysis showed no significant hits in the top 20 predicted sites using NGS, suggesting that the accuracy and safety of our system lends itself as a viable strategy for therapeutics.

The inventors have further herein shown broader applicability of the invention, applying the approach to patients with DMD. To date, treatments that specifically target duplications in DMD have not been extensively studied even though duplications of one or more exons comprise approximately 10% of the DMD mutation spectrum (27). Recent therapeutic strategies for DMD undertaken by other groups include gene replacement therapies, which deliver truncated but functional microdystrophin genes (36, 37). One type of gene replacement therapy is exon skipping, where antisense oligonucleotides complementary to regions of the dystrophin premature mRNA are used to induce skipping of one (38, 39) or more exons (40), hence restoring the open reading frame to produce a shorter dystrophin protein. Similarly, previous studies from other laboratories using CRISPR/Cas9 have demonstrated that this system can be utilized to restore the reading frame of large deletions in the dystrophin gene (20). However, one potential shortcoming of these approaches is that the shorter dystrophin product ameliorates the disease phenotype only to the extent of making them similar to patients with Becker muscular dystrophy, who exhibit expression of a truncated, yet functional dystrophin protein (41). Thus, the data presented herein is of particular importance as removal of a duplication leads to restoration of full-length dystrophin, which represents novel therapeutic opportunities for DMD patients with duplications.

An important consideration in establishing a treatment for DMD is determining how much dystrophin is necessary to ameliorate the disease phenotype. It is estimated that in humans about 20% of truncated dystrophin protein expression is sufficient to have a less severe phenotype and maintain ambulation (42, 43). Furthermore, studies in mdx mice suggest that approximately 5% of full-length dystrophin can improve disease pathology and >20% is needed to fully protect muscle fibers from exercise-induced damage (44-46). The present data demonstrates 4.42% expression of full-length dystrophin accompanied by restoration of components of the DGC.

Here, the inventors have developed a pipeline for genome engineering strategies using easily accessible patient cells and have demonstrated that individually tailored single RNA guides are able to remove large duplicated genomic rearrangements in two different genetic disorders.

In another embodiment, the invention provides compositions and kits comprising one or more of the components for use in the methods of the present invention and optionally instructions for use of same in the methods of the present invention.

In another aspect, the inventors have developed an animal model for studying replicative genetic disorders by successful generating a genetically engineered mouse model comprising a duplication of Dmd exons 18-30 using engineered targeted endonuclease technology. Constructs and methodologies for developing same are also provided as well as uses thereof.

### Definitions

“Administering to the cell(s)” as used herein means both in vitro and in vivo administration to the cells and can be direct or indirect administration, as long as the cells are at some point exposed to the substance being administered.

“Effective Amount” and “Therapeutically Effective Amount” as used herein means an amount effective, at dosages and for periods of time necessary to achieve the desired results. For example, an effective amount of a substance may vary according to factors such as the disease state, age, sex, and weight of the individual, and the ability of the substance to elicit a desired response in the individual. Dosage regimes may be adjusted to provide the optimum therapeutic response. For example, several divided doses may be administered daily or the dose may be proportionally reduced as indicated by the exigencies of the therapeutic situation.

“Pharmaceutically Acceptable Carrier” as used herein means any medium which does not interfere with the effectiveness or activity of an active ingredient and which is not toxic to the hosts to which it is administered. It includes any carrier, excipient, or vehicle, which further includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers, excipient or vehicles include but are not limited to saline, buffered saline, dextrose, water, glycerol, ethanol, and combinations thereof. Such media and agents for an active substance and uses thereof are well known in the art (e.g., “Remington: The Sciences and Practice of Pharmacy, 21^{st} Edition”, (University of the Sciences in Philadelphia, 2005).

“Isolated” refers to a protein or nucleic acid that, if naturally occurring, is in an environment different from that in which it may naturally occur.

The terms “nucleic acid”, “polynucleotide”, “nucleotide sequence”, and “oligonucleotide” are used interchangeably and refer to a polymer of nucleotides of any length, either deoxyribonucleotides or ribonucleotides, or analogs thereof. A polynucleotide may comprise one or more modified nucleotides (e.g. methylated nucleotides and nucleotide analogs). Non-limiting examples of nucleic acids include messenger RNA, isolated DNA of any sequence, isolated RNA of any sequence, guide RNA, recombinant polynucleotides, vectors, probes, and primers.

Herein the term “gene” includes a DNA region encoding a gene product, as well as all DNA regions regulating the production of the gene product, irrespective of whether the particular regulatory sequence is adjacent to the coding and/or transcribed sequences on a chromosome. Accordingly, a gene includes, for example, promoter sequences, terminators enhancers, silencers, insulators, boundary elements, replication origins, matrix attachment sites, locus control regions, and translational regulatory sequences such as ribosome binding sites.

Herein the term “promoter” refers to a sequence of DNA, usually upstream (5’) of the coding region of a structural gene, which controls the expression of the coding region by providing recognition and binding sites for RNA polymerase and other factors which may be required for initiation of transcription.

Herein the term “gene product” includes the direct transcriptional product of a gene (e.g. mRNA, tRNA, rRNA, antisense RNA) or a protein produced by translation of an RNA transcribed from the gene. Gene products further include modified RNAs (e.g. by capping, polyadenylation, methylation, and editing) and modified proteins (e.g. by methylation, etylation, phosphorylation, ubiquitination, ADP-ribosylation, and glycosylation).

The terms “polypeptide”, “peptide”, and “protein” are used interchangeably and refer to chains or polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein, the term “amino acid” refers to an organic acid containing both a basic amino group (NH₂) and an acidic carboxyl group (COOH), and includes natural and/or unnatural or synthetic amino acids, including both the D or L optical isomers, and amino acid analogs.

Herein the term “plasmid” refers to a circular double-stranded DNA construct capable of being used as a vector for introducing DNA into a cell.

As used herein, the term “Cas protein” has its conventional meaning as used in the art where it refers to a product of a Cas gene which in nature is typically coupled to, associated with, or in the vicinity of a CRISPR locus. In one aspect, the term encompasses proteins which in their natural state have DNA cleavage activity (i.e. endonuclease activity). For instance, the term encompasses Cas9 isolated from for example Streptococcus pyogenes and/or Streptococcus thermophilus. In one aspect, the Cas protein directs cleavage of one or both strands of DNA at the location of a target sequence, such as within the target sequence and/or within the complementary sequence of the target sequence (52, 53, 55, 58). It also encompasses conservative amino acid substitutions of native Cas proteins, wherein conservative substitutions do not affect the native endonuclease activity of the Cas proteins.

“Cas proteins” may be part of a fusion protein, for instance a Cas polypeptide is part of a fusion protein comprising one or more heterologous protein domains. Non-limiting examples of protein domains which may be fused with a Cas polypeptide include reporter sequences, epitope tags, and protein domains that have one or more activities such as: transcriptional activating activity (i.e. functioning as trans-activators of transcription), transcription repression activity, methylase activity, histone modification activity, and nucleic acid binding activity. For instance, in some embodiments a “Cas protein” is a Cas9 isolated from S. pyogenes or S. thermophilus and directly fused to a transcriptional activator (e.g. VP160 and/or VP64) at its 5′ end.

Cas proteins may include amino acid deletions or substitutions which alter the sequence of the protein from its natural state. In some embodiments, changes to the amino acid sequence modify protein activity (e.g. enzymatic activity, including endonuclease activity) which in nature is associated with the protein. For instance, a plasmid encoding a Cas protein can be mutated such that when expressed the Cas protein lacks the capacity to cleave one or both strands of a nucleic acid comprising a target sequence. In another aspect the Cas protein is a S. pyogenes Cas9 protein engineered to contain an aspartic acid to alanine substitution at residue 10 (D10A) in the RuvC I catalytic domain of Cas9, which converts the endonuclease to a nickase.

“Endonuclease” is a protein that has DNA cleavage activity, for instance a Cas protein, Fok1 (for instance that complexes with a zinc finger or TALENS).

Herein the term “guide” refers to any polynucleotide sequence or protein capable of forming a complex with an endonuclease and a target nucleic acid sequence. It can refer to any type of guide useful for the present invention including but not limited to a Zinc finger protein or a TALENS array or a guide RNA or a DNA guide.

Herein the terms “guide RNA”, and “gRNA”, are used interchangeably and refer to the polynucleotide sequence capable of forming a complex with an endonuclease such as a Cas protein and has a region that is complementary to a target sequence of a polynucleotide. The guide RNA may consist of a single polynucleotide or multiple polynucleotides complexed together using for example hydrogen bonds between complementary base pairs. The guide RNA comprises the “guide sequence”, which refers to the sequence within the guide, typically about 18-20 bp in length, which hybridizes to the target site. In one embodiment, a gRNA can include a guide sequence complementary to human or mouse nucleic acid, such as the ones used herein in the Examples. The gRNA may be a chimeric or recombinant RNA. In some embodiments, one or more gRNAs are derived from a type I, type II, or type III CRISPR system (56). In some embodiments, at least a portion of one or more gRNAs is derived from an organism comprising an endogenous CRISPR system, such as Streptococcus pyogenes or Streptococcus thermophilus. In general a gRNA is characterized by sequences and structures which promote the formation of a CRISPR complex at a target sequence.

In general “gRNA” (guide RNA) is a fusion of the crRNA and tracrRNA, provides both targeting specificity and scaffolding/binding ability for an endonuclease such as Cas9 nuclease and does not exist in nature. “gRNA sequence”: in general are the nucleotides (.e.g 18-20 nucleotides) that precede the endonuclease recognition site, such as the PAM sequence, in the genomic DNA, what gets put into a gRNA expression plasmid, does not include the PAM sequence.

“tracrRNA”: the endogenous bacterial RNA that links the crRNA to the endonuclease such as, Cas9 nuclease, can bind any crRNA.

“crRNA” as used herein is the endogenous bacterial RNA that confers target specificity, and requires tracrRNA to bind to the endonuclease, such as Cas9.

Herein the terms “target sequence” and “target site” are used interchangeably and refer to a nucleic acid sequence or a polynucleotide to which a guide sequence is complementary (or sufficiently complementary) to bind. It should have, be proximate to or adjacent to an endonuclease recognition site to enable the endonuclease within a guide/endonuclease complex to bind and cleave the genetic material at the desired location. For instance in the Cas9, the endonuclease target site is a PAM sequence which is within the genomic DNA and adjacent to the target sequence for the guide but not within the guide sequence. The presence of an endonuclease recognition site is important in determining suitable target sequences and in designing and optimizing guides.

Full (100%) complementarity between the guide, e.g. gRNA, and the target sequence is not necessarily required, so long as the guide sequence and target sequence are sufficiently complementary to cause hybridization and promote formation of an endonuclease/guide/target complex, e.g. a CRISPR complex. A target sequence may comprise either DNA or RNA, and in some embodiments may be located in the nucleus, cytoplasm, or organelle (e.g. mitochondria or chloroplast) of a cell.

Herein the term “endonuclease/guide/target” encompasses a guide comprising a region capable of hybridizing or coupling with a target sequence and a region capable of forming a complex with an endonuclease complexed with the endonuclease and a hybridized to the target sequence. Thus “CRISPR complex” as used herein encompasses a guide RNA comprising a region having a guide sequence and a region capable of forming a complex with a Cas complexed to one or more Cas proteins and hybridized to a target sequence.

Herein the terms “patient”, “subject”, and “individual” are used interchangeably and refer to a vertebrate, preferably a mammal, and more preferably a human. In some aspects of the invention, tissues and cells of a biological entity such as from a patient are obtained in vivo or cultured in vitro.

Off-target effects refers to guides, such as gRNA binding to target sequences that do not match exactly, causing the endonuclease, such as Cas9 to function in an unintended location causing effects.

ORF: Open Reading Frame, the codons that make up a gene.

PAM: Protospacer Adjacent Motif, required sequence for endonuclease recognition of the CRISPR/CAS system that must immediately follow the gRNA recognition sequence but is NOT in the gRNA.

sgRNA: single guide RNA, the same as a gRNA, which is a single stranded RNA.

Herein the term “muscular dystrophy” includes all forms of dytrophin-deficient muscular dystrophy. Non-limiting examples include Duchenne muscular dystrophy and Becker muscular dystrophy. More particularly it refers to the subset of said dystrophies caused by duplicate genetic material.

Herein the term “muscle cell” refers to any type of myocyte including cardiac, skeletal, and smooth muscle cells, as well as their progenitor myoblasts, as well as muscle stem cells, called satellite cells.

### Methods of the Invention

In one aspect, the invention provides a method for removing replicated genetic material present head to tail on a nucleotide sequence of a cell, such as a eukaryotic cell with or without an intervening sequence in-between the replicated genetic material. In some embodiments the genetic material is duplicated, in other embodiments it may be present in triplicate or more.

In some embodiments, the method comprises:
(i) delivering to the cell one exogenous polynucleotide guide that binds to a target sequence at each copy of said target sequence within said replicate (e.g. duplicate) genetic material and wherein said target sequence at each occurrence has or is sufficiently proximal to an endonuclease recognition and cleavage site to enable an endonuclease complexed with the guide to complex with or bind the recognition site. In some embodiments, delivery to the cell of the exogenous polynucleotide guide comprising delivering a nucleotide sequence encoding same to the cell for expression within the cell. In other embodiments it can be delivering to the cell the guide per se to the cell;
(ii) delivering to the cell an endonuclease that can complex with the guide and recognizes the endonuclease recognition site, wherein delivering to the cell can include in some embodiments, delivering to the cell a nucleotide sequence encoding the endonuclease to the cell for expression within the cell. In other embodiments it can be delivering to the cell the endonuclease per se. In another embodiment, if the endonuclease is endogenous to the cell (for instance via a transgene), it means supplying conditions to express the endonuclease within the cell; and wherein
the delivery of the guide and endonuclease is done in a manner so that they form an endonuclease guide complex where upon the guide binds to the target sequence, the endonuclease recognizes the endonuclease recognition site and creates a double stranded break at each cleavage site, thus removing the duplicate genetic material.

In some embodiments the exogenous polynucleotide guide and endonuclease can be delivered to the cell by delivering their coding sequence to the cell in one (i.e. on the same vector) or more vectors, such as plasmids, phages, cosmids or protein. In other embodiments they can be delivered as formed endonuclease/guide complex, via suitable delivery means, such as liposomes, electroporation or other suitable means known in the art.

“One exogenous polynucleotide guide” as used herein refers to one type of polynucleotide guide or a guide that targets one target sequence. The guide may be present in multiple copies within the cell or multiple copies of its coding sequence may be delivered to the cell or present within the vector delivered to the cell. The present invention only requires a guide that targets only one target sequence, which can include multiple sites where that target sequence occurs, for instance in replicated genetic material.

In other embodiments, the method of further comprises the step of repairing and joining the cleaved nucleotide sequence. This can be done by non-homologous end joining or homology directed repair, but preferably non-homologous end joining and wherein the method of the invention results in at least one less copy of the genetic material or resulting in one copy of the genetic material where before there was two or more. In some embodiments, other cellular repair mechanisms may also be used, such as microhomology repair.

In some embodiments, the the exogenous polynucleotide guide is delivered to the cell by:
(i) transfecting or transducing the cell with a vector comprising nucleic acid encoding the guide;
(ii) if the endonuclease is exogenous, transfecting the cell with a vector nucleic acid encoding the endonuclease,

wherein the cell expresses the guide and the endonuclease, the guide forming a complex with the endonuclease, and wherein the vector in (i) and (ii) can be the same or different. In some embodiments, the vector is a plasmid or a viral particle or protein. In other embodiments, it is a plasmid or viral particle.

In some embodiments, the replicative or duplicate genetic material is associated with a disease, such as Duchenne's muscular dystrophy that is caused by duplicative genetic material, such as duplication of exons 18-30 or MECP2 duplication syndrome. causing variant and the method is used in the prevention or treatment of said disease by administering to the patient, or embryo or egg a guide and endonuclease or vector coding same or guide/endonuclease complex alone or together of the invention as per the methods herein before described.

In one embodiment, the invention provides a method for designing an exogenous polynucleotide guide for use in the method of the invention, said method comprising:
(i) sequencing the duplicate material and the junction,
(ii) identifying an endonuclease recognition and cleavage site within said duplicate material;
(iii) selecting a target sequence with minimal off targets and that has or is proximal to the endonuclease recognition and cleavage site to enable an endonuclease complexed with the guide to recognize and cleave the duplicate genetic material;
(iv) designing the guide to bind to said target sequence and complex with the endonuclease.

In another aspect, the invention provides a composition comprising:

(a) one or more of the following:
a nucleic acid encoding one guide, such as a single guide RNA of the invention (wherein in one embodiment the composition comprises one or multiple copies of the nucleic acid encoding the guide, such as a single guide RNA);
(i) a nucleic acid encoding an endonuclease, such as a Cas protein (such as Cas9) or a Cas fusion protein;
(ii) an endonuclease such as a Cas protein (such as Cas9) or a Cas fusion protein;
(iii) a guide, such as a guide RNA of the invention (wherein in one embodiment the composition comprises one or multiple copies of the one guide);
(iv) an endonuclease Cas or Cas fusion protein of the invention complexed with the guide RNA of the invention;
(v) both (iii) and (iv);
(vi) a vector comprising (i) and (ii) or two vectors comprising (i) and (ii) respectively and
(vii) any one or more of the above and optionally a delivery vehicle, such as a bacterial (e.g. plasmid), viral (e.g. phage), cosmid, or other delivery vehicle (for instance in the case of delivery of (iii)-(vi) could be packaged in a liposome); and optionally

(b) a pharmaceutically acceptable carrier.

In another embodiment, the invention provides a use of the compositions in the methods and uses of the invention. In one embodiment the components or compositions of the invention can be in a kit comprising one or more of same and optionally instructions for their use in carrying out any one of the methods of the invention.

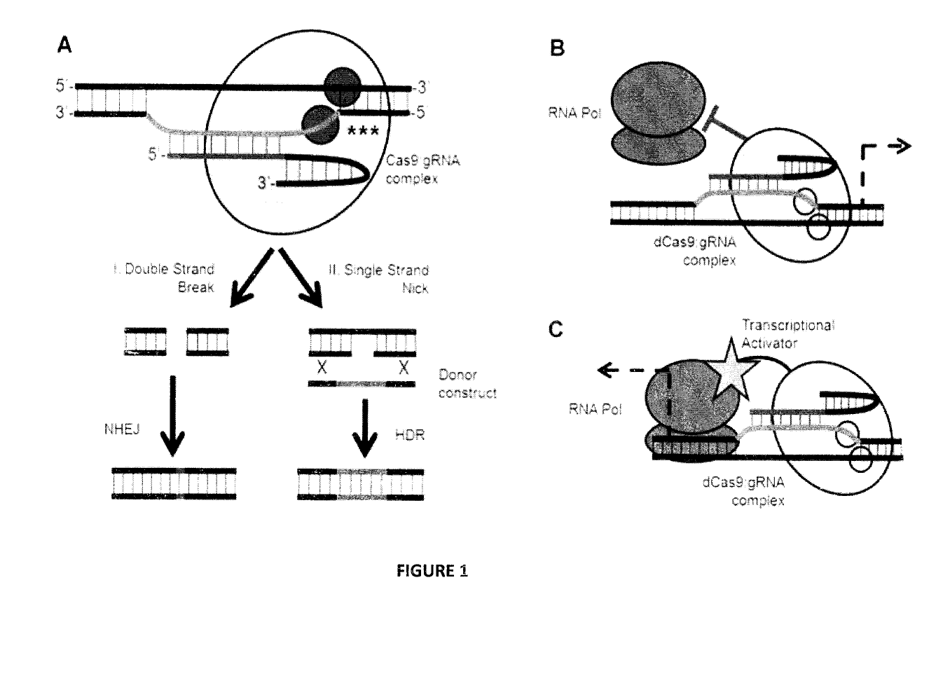

Referring to FIG. 1, In one aspect of the invention, a single guide RNA (sgRNA), consisting of a crRNA sequence that is specific to the DNA target, and a tracrRNA sequence that interacts with the Cas9 protein, binds to a recombinant form of Cas9 protein that has DNA endonuclease activity. The resulting complex will cause target-specific double-stranded DNA cleavage. The cleavage site may be repaired by the non-homologous end joining (NHEJ) DNA repair pathway.

CRISPR loci in a bacterium contain “spacers” that in type II adaptive immune systems were created from invading viral or plasmid “protospacer” DNA. On subsequent invasion, Cas9 nuclease attached to tracrRNA: crRNA is guided to the invading protospacer sequence, but Cas9 will not cleave the protospacer sequence unless there is an adjacent PAM sequence. The “spacer” in the bacterial CRISPR loci will not contain a PAM sequence, and will thus not be cut by the nuclease. But the protospacer in the invading virus or plasmid will contain the PAM sequence, and will thus be cleaved by the Cas9 nuclease. For editing mammalian genes, guideRNAs (gRNAs) are synthesized to recognize mammalian gene sequences having a PAM sequence at the 3′-end.

For Cas9 to successfully bind to DNA, the target sequence in the genomic DNA must be sufficiently complementary to the gRNA sequence for the gRNA to bind with it and must be immediately followed by the correct protospacer adjacent motif or PAM sequence. The PAM sequence is present in the DNA target sequence but not in the gRNA sequence. Any DNA sequence with the correct target sequence followed by the PAM sequence will be a Cas9 recognition and cleavage site and will be bound by Cas9. The presence of the target sequence without the PAM following it is not sufficient for Cas9 to cut. Further, the presence of the PAM sequence alone is not sufficient for Cas9 to cut.

The PAM sequence varies by the species of the bacteria from which the Cas9 was derived. The most widely used Type II CRISPR system is derived from S. pyogenes and the PAM sequence is NGG located on the immediate 3′ end of the gRNA recognition sequence. The PAM sequences of other Type II CRISPR systems from different bacterial species are listed in the Table below. It is important to note that the components (gRNA, Cas9) derived from different bacteria will generally not function together. Example: S. pyogenes (SP) derived gRNA will not function with a N. meningitidis (NM)derived Cas9.

In one embodiment, of using the CRISPR/Cas9 engineered system both the gRNA and Cas9 is expressed in target cells. The respective promoters for Cas9 and gRNA expression will ultimately determine the species specificity of a particular system. CRISPR cassettes can either contain both gRNA and Cas9 expressing cassettes on a single plasmid or they can be expressed from two separate plasmids.

In one embodiment, the present invention provides a method of restoring wild type gene expression in vivo or in vitro in a eukaryotic cell comprising using one single guide RNA and an endonuclease, such as a Cas protein bearing effector domains to modulate expression in vivo or in vitro in eukaryotic cells.

In one aspect the method in a eukaryotic cell (e.g. skeletal or cardiac muscle cell) comprises:
(i) transfecting the eukaryotic cell with a nucleic acid encoding one single guide RNA, which comprises a region complementary to genomic DNA of a target region of the eukaryotic cell (i.e. the target sequence) and a region that can interact and complex with an endonuclease Cas protein;
(ii) transfecting the eukaryotic cell with a nucleic acid encoding an endonuclease Cas protein that can complex with the RNA;

wherein the eukaryotic cell expresses the RNA and the endonuclease Cas protein, the endonuclease Cas protein complexes with the RNA, the RNA binds to the complementary genomic DNA of the target region of the gene (i.e. the target sequence).

In one embodiment, RNA complementary to genomic DNA of the target region of includes RNA that is complementary to all or part of a target sequence. In one aspect the RNA binds to a specific 18-20 nt long DNA sequence, however length can be different for different Cas9 species or endonucleases. In one aspect the RNA does not need to be 100% complementary to the target sequence of the target region, but is sufficiently complementary to enable base pair binding to the target sequence to enable the Cas protein fusion protein to bind to the target region.

In one embodiment, the guide RNA comprises a region that is 18-20 nt in length (i.e. the “guide sequence”; see definition above) that is complementary to genomic DNA of the target region, and a region that can interact and complex with an endonuclease Cas protein.

In one embodiment, each gRNA is a single chimeric guide RNA comprising a region having a guide sequence (e.g. a sequence complementary to a target sequence) and a region which interacts with an endonuclease Cas protein. In another embodiment the gRNA comprises of multiple RNAs (e.g. a tracrRNA and crRNA), wherein one of the RNAs comprises the guide sequence complementary to the target region and one or more of the RNAs provides a region to interact with the endonuclease Cas protein (see e.g. 9).

In one embodiment the nucleic acid encoding the guide RNA is inserted into a vector suitable for transfection or transduction into the eukaryotic cell and subsequent transcription within the cell. In one embodiment the vector is selected from the group of vectors consisting of: plasmids, recombinant adeno-associated viral (rAAV) or lentiviral particles.

The invention contemplates the use of any gRNA which contains a guide sequence complementary to a desored target polynucleotide. In one embodiment only one gRNA is used in the method.

In one embodiment the DNA encoding the endonuclease Cas protein that interacts and can complex with the gRNA. In another embodiment it is a codon-optimized version of the Cas protein, optimized for the respective eukaryotic cell. In one embodiment the Cas protein is human codon optimized.

The invention contemplates the use of any Cas protein capable of complexing with a gRNA to target a polynucleotide. In certain embodiments, the Cas protein may be a derivative of a naturally occurring Cas protein. The term “derivative” encompasses amino acid sequence variants of a polypeptide, covalent modifications, and fusions thereof. Suitable derivates of Cas polypeptide or a fragment thereof include but are not limited to fusions, mutants, and covalent modifications of a Cas protein or a fragment thereof. A Cas protein, which includes a derivative of a Cas protein, may be obtained from a cell, synthesized chemically, or by a combination of these procedures. The cell may naturally produce Cas protein, or may not naturally produce Cas protein but be engineered to produce Cas protein by one or more exogenously introduced nucleic acids. In other cases the cell may naturally produce Cas protein and be genetically engineered to produce the endogenous Cas protein at a higher expression level than naturally occurs. Non-limiting examples of Cas proteins and complexes which may be used with the present invention include forms of Cas9, Csm/Cas10, Cas3, and one or more proteins of the CRISPR-associated complex for antiviral defense (Cascade), including Cse1, Cse2, Cas7, Cas5, Cas6e, Csy1, Csy2, Csy3, and Cas6f. In some embodiments, multiple activated Cas proteins of a Cas protein complex are used in combination with one or more gRNAs.

The invention contemplates any construct that is capable of containing a coding sequence for Cas9. In one embodiment the vector is selected from the group of vectors consisting of: plasmids, rAAV, and lentiviral particles.

In one embodiment the nucleic acid encoding the single gRNA and the endonuclease Cas protein is DNA. In one embodiment they are on one vector. In another embodiment the DNA encoding the gRNA and the Cas protein are on different vectors. In one embodiment the vector comprises more than one copy of the nucleic acid encoding the gRNA and/or the endonuclease.

In one embodiment components (i) and (ii) of the method noted above are co-transfected at the same time in one composition or proximal in time in different compositions.

In yet another embodiment the components (i) and (ii) of the method noted above are inserted into a suitable delivery system or vehicle, alone or together, such as an adenovirus for transfection of the mammalian cell, rAAV, lentivirus, or nanoparticles.

In yet another embodiment the method contemplates the exogenous expression and isolation of the Cas fusion protein as herein described complexed in vitro with the gRNA as also herein described, and using a composition comprising said complex in a suitable delivery vehicle for transfecting same into the eukaryotic cell.

In one embodiment the eukaryotic cell is selected from the group of cells from humans, mice, dogs, and other species as desired. In one embodiment it is a mammalian cell. In another embodiment the mammalian cell is selected from the group of mammalian cells consisting of: human, mouse and dogs. In one embodiment the cell is a human cell.

In another embodiment the cells are mammalian cells selected from the group consisting of:

oocytes, myoblasts, myocytes and cardiomyocytes.

In one embodiment the invention comprises a cell transfected using the methods of the invention described herein.

### Therapeutic Applications and Compositions

The invention can be used in the treatment or therapy of conditions that are caused by replicative or duplicate genetic material such as CNVs, such as in certain patients with DMD or MECP2 duplication syndrome.

### Muscular Dystrophy

Muscular dystrophy is a group of muscle disorders which weaken the musculoskeletal system and impair locomotion. Muscular dystrophies are characterized by defects in muscle proteins, death of muscle cells and tissues, and progressive impairment of muscle movement. Patients severely affected by muscular dystrophy may have cognitive impairment, behavioural, vision, and speech problems (59). Muscular dystrophies are generally inherited and are associated with mutations in different genes generally encoding muscle effector proteins. There are many forms of muscular dystrophy including but not limited to Becker, limb-girdle, congenital, facioscapulohumeral, myotonic, oculopharyngeal, distal, and Emery-Dreifuss muscular dystrophy.

Duchenne muscular dystrophy (DMD) is the most common form of muscular dystrophy and is inherited in an X-linked autosomal recessive form. The disease is caused by mutations in the dystrophin gene (Dmd) that result in a lack of functional dystrophin. In some patients it is caused by duplicate genetic material.

A method of the invention contemplates the use of a single guide and an endonuclease such as gRNAs and Cas fusion proteins described herein in the treatment of a condition in a mammal that can benefit from removal of duplicate genetic material, such as DMD, the method comprising administering to said mammal an effective amount of:
(i) a composition comprising a nucleic acid encoding one single guide RNA complementary to genomic DNA of target region of the eukaryotic cell of said mammal and a region that can complex with the endonuclease Cas protein; and
(ii) a composition comprising a nucleic acid encoding an endonuclease Cas protein that can complex with the RNA;

wherein the nucleic acids of (i) and (ii) are incorporated into a vector (either a single vector or separate vectors) and in a delivery vehicle, as applicable, suitable for delivering same to the eukaryotic cell of the mammal in a manner that enables the cell to be transfected with said nucleic acids and express same once transfected.

In another embodiment of the invention, the method comprises administering to a subject in need cells that express or comprise the gRNA and Cas fusion proteins of the invention. In one embodiment, the method comprises harvesting cells from the patients, transfecting the cells in accordance with the present invention to obtain cells wherein expression of the duplicate gene to be restored to wild type expression and administering said modified cells to said patient.

In another embodiment the patient is monitored for expression of the gene in question. In another embodiment the method of the invention is repeated as needed for said patient.

In one embodiment the eukaryotic cells are myoblasts or myocytes.

In one embodiment the gRNA and the Cas fusion proteins of the invention or nucleic acids encoding same can be in one composition or multiple compositions. In another embodiment only one gRNA is used. In another embodiment the compositions of the invention comprise a suitable pharmaceutical acceptable carrier.

In one embodiment the invention provides a composition comprising:

(a) one or more of the following:
(i) a nucleic acid encoding one single guide RNAs as described herein;
(ii) a nucleic acid encoding a Cas fusion protein as described herein;
(iii) one or more Cas fusion proteins as described herein complexed with a gRNA as described herein;
(iv) both (i) and (ii); and

(b) optionally a pharmaceutically acceptable carrier.

In another embodiment the invention provides a kit comprising one or more compositions of said invention, and optionally instructions for their use in carrying out any one of the methods of the invention.

An advantage of the present invention is that it can be implemented to treat or prevent conditions caused by duplicate genetic material such as CNVs using only one single guide or gRNA (i.e. as opposed to more than one single guide RNA that has different targets). This makes it easier and less complex solution then using, designing and making multiple guides.

### Pharmaceutical Compositions and Modes of Administration

The guides and endonucleases, such as the single gRNAs and Cas fusion proteins of the invention and/or the nucleic acids encoding same can be administered by any means that produce contact of the gRNAs and Cas fusion proteins with the target elements of the cell to cut the target genetic material in vitro or at the desired sites of action in the body of a patient to produce a therapeutic effect, in particular a beneficial effect, and in one embodiment a sustained beneficial effect. The compositions of the invention comprising the one or more nucleic acids encoding the gRNAs, the nucleic acid encoding the Cas fusion proteins of the invention, and/or the Cas fusion proteins complexed with the gRNA can be administered simultaneously or sequentially and in any order at different points in time to provide the desired beneficial effects. A compound and composition of the invention can be formulated for sustained release, for delivery locally or systemically. It lies with the capability of a skilled person, such as a physician or veterinarian to select a form and route of administration that optimizes the effects of the compositions and treatments of the present invention to provide therapeutic effects, in particular beneficial effects.

In one aspect the invention includes administration of the Cas fusion protein and/or gRNA or nucleic acids encoding same to the site of action—directly or through a mode of delivery (e.g. sustained release formulations, delivery vehicles, such as liposomes) that results in delivery or site-directed delivery of the guide and endonuclease (peptide, gRNA or encoding nucleic acids for the guide and endonuclease) to a particular cell or site in the body. This would include but is not limited to the use of a polynucleotide encoding an endonuclease (such as Cas or Cas fusion protei and/or a guide (such as gRNA), e.g. via gene therapy or through an expression system in vitro, ex vivo or in vivo, as the case may be that results in expression of the endonuclease and guide, e.g., Cas fusion protein and/or gRNA, and subsequent creation of the endonuclease/guide/target complex.

The above described substances including Cas fusion protein(s), gRNAs, and nucleic acids encoding these substances may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals.

Thus in one embodiment, the invention provides the use of Cas fusion protein and/or gRNAs and/or nucleic acids encoding same in the preparation of a medicament for the treatment of muscular dystrophy, such as Duchenne muscular dystrophy in that portion of patients whose disease is caused by duplicate genetic material. In one embodiment, a therapeutically effective amount of same or a pharmaceutical composition as described herein is administered to a patient in need thereof. A patient in need thereof is any animal, in one embodiment a human, that may benefit from the effect of these substances in the treatment of a condition that may benefit from removal of duplicate genetic material.

The compositions of the present invention may be administered in a convenient manner such as by injection (subcutaneous, intravenous, etc.) or oral administration. Depending on the route of administration, the active substance may be coated in a material to protect the components of the composition from the action of enzymes, acids and other natural conditions that may inactivate same. In one embodiment, the compositions of the invention are administered directly or proximate to the desired site of action, by injection or by intravenous administration.

As will also be appreciated by those skilled, administration of substances described herein may be by an inactive viral carrier, such as AAV. In one embodiment the AAV is naturally occurring. In one embodiment the AAV is selected from the group consisting of AAV6, AAV8, and AAV9. In one embodiment the AAV is recombinant. In one embodiment the AAV is a DJ serotype. In one embodiment a Cas fusion protein and/or gRNA and/or a nucleic acid encoding same can be administered in a vehicle comprising saline and acetic acid.

As such, in one embodiment, systemic injection of viral particles (AAV viral particles) comprising the vector encoding the Cas9 and the single guide RNA for transcription in vivo in tissue can be used.

Further, in one embodiment, a Cas fusion protein and/or gRNA, nucleic acids encoding same, vectors comprising same, and vehicles comprising any of the foregoing, may be administered in a form that is conjugated to another molecule or compound, including a peptide, to facilitate delivery to a desired site, or in a vehicle, e.g. a liposome or other vehicle or carrier for delivery.

### General Background on Guide RNAs and Testing

To develop guide RNAs, each target sequence (about 18 to 20 bp) is followed by an S. pyogenes Cas9 specific proto-spacer adjacent motif (PAM) sequence (NGG). If the guide sequence did not begin with a 5′-G, a G nucleotide was added to the primer to optimize gRNA expression.

DNA sequences encoding the guide RNAs were inserted into vectors such as plasmids, for subsequent transfection into the target cells.

This construct can be incorporated into AAV8 viral particles which can be systemically injected in vivo in tissue to be taken up by the cells and affect genetic editing in vivo.

The following protocol can be used to study effects of systemic rAAV administration:
(i) injection of 10¹¹ of the viral particles intravenously (e.g. into the tail vein) to mice with the relevant condition (or model of the condition) with the genetic material to be corrected to study effects of systemic rAAV administration; and
(ii) analysing the effect of same by one or more of the following:
   (1) analyzing the effect on relevant gene expression by immunoblotting and immunofluorescence analysis;
   (2) conducting functional tests, such as for muscular dystrophy testing muscle strength of hindlimbs and forelimbs by grip strength test measurements using BIO-GS3 instrument from BIOSEB; and/or calculating muscle regeneration index in TA, gastrocnemius, EDL, triceps and diaphragm by counting numbers of myofibers with centrally located nuclei.

Taken together, these experiments can be used to evaluate rAAV-mediated genetic editing and whether it provides therapeutic benefit.

### Genetically Engineered Animals Comprising Duplicate CNVs

In one aspect genetically engineered animals comprising duplicative CNVs can be generated which are useful as animal models for a particular disorder related to the duplicative CNV.

The method comprises generating guides, in one embodiment at least two guides, in another embodiment four guides, towards either end of the intended duplication that are delivered together with the endonuclease to embryonic stem cells (e.g. by incorporating the coding sequence of the guides and endonuclease into plasmids using the same plasmid or separate plasmids) and electroporation into the cell or by other means), selecting successful clones that have incorporated the duplication and expanding same. Aggregating the clones into a blastocyst of a pregnant foster animal such as a mouse and crossing the resulting chimeras to establish a germline transmission of the genetically engineered mouse with the duplication. In one embodiment a dup18-30Dmd mouse was generated using four sgRNAs and the CRISPR/cas9 system. In one embodiment the guide is a nucleic acid, such as RNA and its coding sequence is incorporated into a plasmid, phage or other vehicle for expression in the cell. In one embodiment, the endonuclease can be delivered to the cell as a protein through suitable delivery means, as long as the guide and endonuclease are present together within the cell to enable it to form an endonuclease/guide complex and endonuclease/guide/target complex for creation of the duplication within the cell and incorporation of same within the genome of the cell.

As such, the invention also provides constructs for forming said genetically engineered animal comprising plasmid, phage, cosmid or other vehicle encoding a desired guide and a vehicle, the same or different as the one encoding the guide, comprising the coding sequence for the endonuclease, wherein the vehicle once administered to the cell expresses the guide and endonuclease and results in formation of the endonuclease/guide and endonuclease/guide/target complex. In another embodiment, the invention comprises compositions comprising the vehicle or vehicles expressing the guide and endonuclease. In another embodiment the invention provides a composition comprising the guide/endonuclease complex for administration to the cell. Such a complex can be delivered to the cell through a suitable delivery vehicle such as a liposome or the like. In another embodiment the invention provides kits for forming a genetically engineered mouse comprising one or more of the constructs or complexes required to generate the desired animal and optionally instructions for same.

The invention also provides methods and uses of said animal models and constructs in the formation of new genetically engineered animals, in research, drug and treatment design, including guide design and endonuclease selection and optimization, wherein the effects of any of the foregoing can be monitored by the effect on the animal, the restoration of function where there was dysfunction, and to study effects on the genome, expression of peptides or genetic material and the like.

The present invention is described in the following Examples, which are set forth to aid in the understanding of the invention, and should not be construed to limit in any way the scope of the invention as defined in the claims which follow thereafter.

### EXAMPLES

Here, the inventors have established a pipeline using easily accessible patient cell lines as a proof of the methodology and tools of the present invention and to show that an engineered targeted endonuclease technology (endonuclease together with one guide) can be used to target replicative genetic material, such as a large tandem X-chromosomal duplication including the MECP2 gene and a large duplication of the dystrophin gene in muscle cells of a patient with Duchenne Muscular Dystrophy (DMD), to remove the duplication and restore wild-type function of the affected gene.

More specifically, the examples shows the use of CRISPR technology for large genomic copy number variations (CNV's)(50, 51), however, the invention is not limited to the use of CRISPR technology or the Cas9 endonuclease, it is used as an example. The examples show that one can utilize the CRISPR/Cas9 system to successfully remove a large 278 kb tandem duplication in fibroblasts of a patient with MECP2 duplication syndrome. Furthermore, it is demonstrated that a modified CRISPR/Cas9 strategy involving a single guide approach successfully removes a 145 kb (exons 18-30) duplication in the DMD gene leading to restoration of full-length dystrophin expression in patient myotubes. This is illustrative and proof that the methods of the claim to remove a replicative genetic material or nucleotide sequence using an engineered targeted endonuclease technology works.

Taken together, the studies show that a targeted engineered endonuclease technology such as the CRISPR/Cas9 system can be adapted to target a variety of inherited disorders caused by large genomic rearrangements.

### Example 1

### Methods

Cell Culture. Fibroblasts from a healthy individual and a patient harbouring a 18-30 exon duplication in DMD (Patient 1) and MECP2 duplication syndrome (Patient 2) were obtained from skin biopsies (skin tags) and established at SickKids pathology laboratory. They were maintained in High Glucose Dulbecco's Modified Eagle's medium (DMEM) (Life Technologies) supplemented with 10% FBS (Life Technologies), L-Glutamine (Life Technologies), 1X penicillin/streptomycin (Life Technologies) at 37° C. with 5% CO₂ incubation. The research ethics boards of each institution approved all of the experiments.

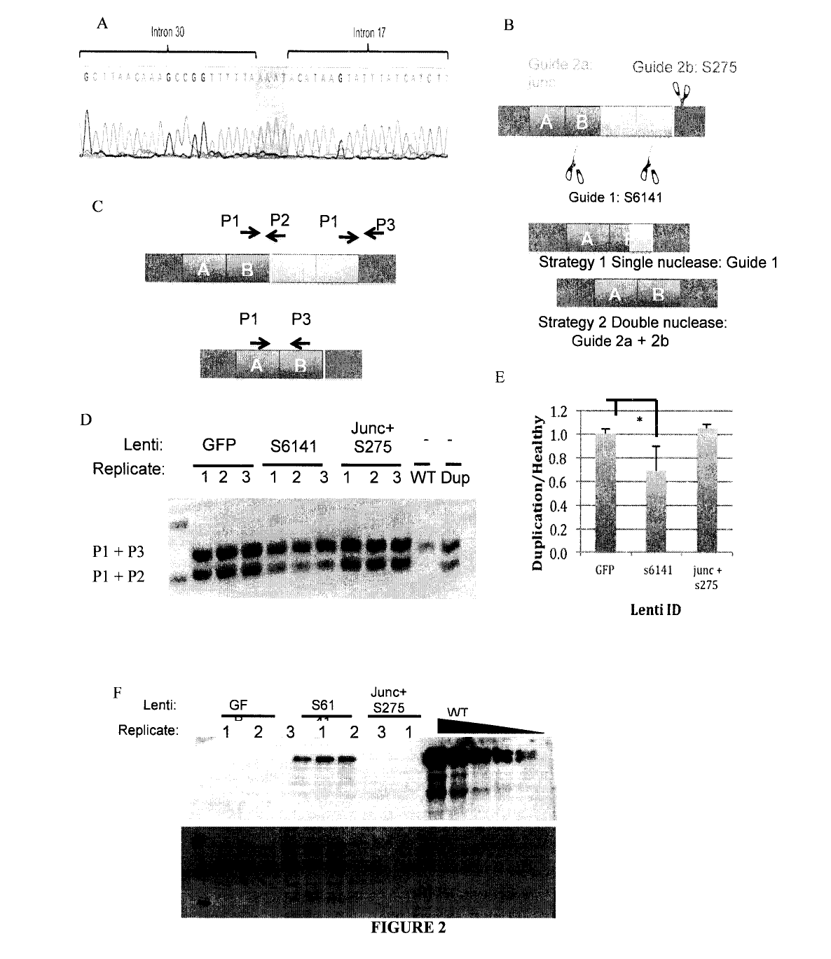

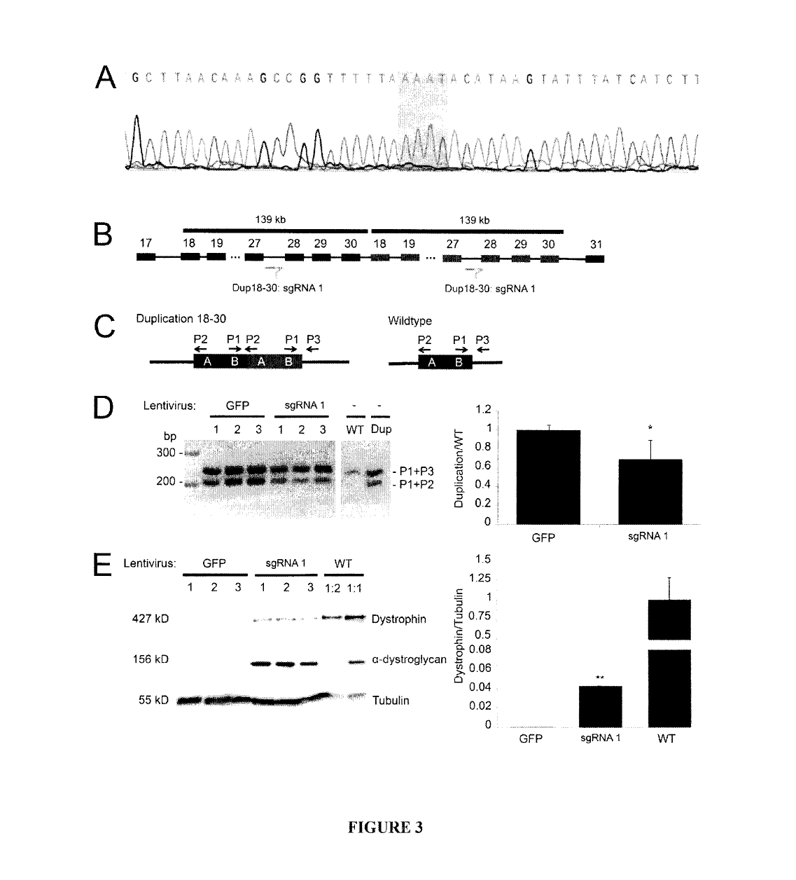

Duplication Junction Mapping. A series of probes near the junction were designed and qRT-PCR followed by sequencing was used to map out the exact break point of the duplication. MECP2 duplication forward primer 5′-CCCACAGAGTAGAGTGGAGCAG-3′ (P1, mecp2 dup common) and reverse 5′-TTAGACAGAGTCTCACTCCATCACC-3′(P3, mecp2 dup out). DMD Dup 18-30 primer forward 5′-CAGCATCATGACCTGTTTCAATC-3′ (P1, dup 18 -30 common f) and reverse 5′-TTGTTAGAGGGCAGCAAGTTTGT-3′ (P3, dup18-30 bound r). (See Table 2 and FIGS. 2 and 3, where P2 junction primers were also made for each of MECP2 and DMD Dup 18-30 for the three way PCR based detection of deletions).

sgRNA Design. All intronic regions within the duplication were analyzed to find the computationally predicted the most active sgRNAs (23). All sgRNAs with a predicted activity score greater than 0.75 were next analyzed using CRISPR Design Tool (26) and ranked according to the least possible number of potential off-target sites. The 3 best predicted sgRNAs (Table 1, also see Table 3 where the extended table of off target analysis is provided) were then subcloned into the Cas9 nuclease plasmid pSpCas9(BB)-2A-GFP (PX458) (Addgene #48138) (46) or LentiCRISPR 2 vector (Addgene #52961) (48). Each plasmid contained a single locus-specific sgRNA in conjunction with SpCas9.

### Nucleoporation

In one method, fibroblasts can be nucleoporated for instance, using a total of 3 μg of DNA using program U-023 on the Amaxa system and the Primary Fibroblast Kit (Lonza). Cells can then be Fluorescence-activated cell sorted 4 days after nucleoporation and DNA was collected on the same day.

Lentivirus production. Lentiviral particles were generated by transfecting 293T-AAV cells (Agilent) with 10 ug of lentiCRISPR 2 plasmid containing the indicated guides, 7.5 ug of psPAX2 (Addgene #12260), and 5 ug pCMV-VSV-G (Addgene #8454). Viral supernatants were collected 60 hours post transfection, centrifuged at 3000 rpm for 10 minutes, and filtered through 0.45 um low-binding filter (48).

Transduction. Two duplication removal strategies were tested. In the first strategy, Cas9 is guided by a single sgRNA, and in the second two different guides one is specific to the junction of the duplication, and the other is just outside of the duplication (FIG. 2B). Patient 1's fibroblasts were transduced with adeno-MyoD (Vector Biolabs) at 5 multiplicity of infection (“MOI”) to induce differentiation into myoblasts. Simultaneously, they were transduced with lentiviral vector containing the coding sequences for the guides: single (S6141; Strategy 1) or two (Junction and S275; Strategy 2) guide(s). Three days later, 2 ug/ul of puromycin was added to enrich for cells containing the lentiCRISPR 2-guide constructs and DNA was collected 7 days post puromycin selection. The cells were further differentiated towards myotubes and proteins were collected 7 days post-differentiation. For MECP2 duplication removal strategy 1 was only tested with either guide 75 or guide 80. Fibroblasts from MECP2 duplication syndrome were transduced with 1 mL LentiCRISPR containing MECP2: sgRNAs 1 (guide 75) or 2 (guide 80). DMD duplication patient fibroblasts were co-transduced with Ad-MyoD (Vector Biolabs) at 100 MOI in DMEM with 1% FBS to induce differentiation of fibroblasts into myoblasts and with a LentiCRISPR.

### Three-Way PCR Based Detection of Deletions

10 ng of DNA was amplified using Multiplex PCR mix (Qiagen) using three different primers. Primers 1 and 3 (P1, P3) are universal to the region of interest, whereas Primer 2 (P2) is specific to the junction of the duplicated region. PCR amplification using combination of P1, P2, and P3 in patient DNA will results in two bands, which correspond to P1-P2- and P2-P3-derived amplicons, respectively. In contrast, only P1-P2-derived amplicon will be observed in healthy control. Primer sequences are shown in Table 2.

### SDS-PAGE and Western Blot

The cells were lysed using RIPA buffer (50 mM Tris HCl pH 7.4, 150 nM NaCl, 1 mM EDTA, 1% deoxycholate, 1% NP40 and 1% Triton X-100 supplemented with Phosphatase and Protease inhibitor cocktails (Roche) and the protein concentration was measured using the BCA assay. 25 μg of of protein lysates were resolved on 3-8% Tris-acetate gels (Novex, Invitrogen) and transferred onto nitrocellulose membranes overnight. The membrane was then incubated in Ponceau S to check for equal loading, blocked in 5% milk/TBST for 1 hour, and probed for dystrophin (MAB1692, Millipore) and β-tubulin (Cat # 05-661, Millipore) overnight at the concentration of 1:200 and 1:2500, respectively. Secondary anti-mouse and -rabbit IgG HRP antibodies were used at the concentration of 1:2500. The signal was detected using SuperSignal West Femto ECL at 1:5 dilution (Life Technologies) and imaged using Bio-Rad Gel Doc imaging system and probed for dystrophin (MAB1692, Millipore), β-dystroglycan (MANDAG clone 7D11, DSHB), α-dystroglycan (kindly provided by Kevin Campbell) and β-tubulin (SantaCruz).

### Next-Generation Sequencing

Off-target analysis was conducted as follows for all lenti-based delivery gene editing experiments. Primers targeting loci corresponding to each sgRNA's top 20 off-target hits, as computed by the CRISPR Design Tool (26), were designed and used to amplify DNA from each gene editing experiments using GeneRead DNAseq Targeted Panels (Qiagen). ˜200 bp amplicons were purified using magnetic beads and library preparation was conducted at The Centre for Applied Genomics (TCAG) at The Hospital for Sick Kids with sample-specific barcodes using the IonTorrent Library preparation kit (Life Technologies). Sequencing was performed using the Ion Torrent Proton. Each potential off-target site was evaluated after aligning corresponding sequencing reads to the Human reference genome (hg19). The proportion of reads that match the reference genome versus those with insertions, deletions and substitution near predicted cleavage sites will be used to estimate the off-target activity of a corresponding single sgRNA.

### Example 2

### CRISPR/Cas9-Mediated Removal of DMD Exons 18-30 Duplication

Given the successful removal of a large chromosomal duplication as per Example 4 below, a similar, single sgRNA approach was applied to large exonic duplications in the DMD gene. Duplications of one or more exons comprise approximately 10% of the DMD mutation spectrum (27).

In DMD patient 1, the inventors determined that the exons 18-30 duplication is a chrX:32,552,206-32,413,149 (hg19) direct tandem repeat in head to tail orientation and sequencing of the duplication junction revealed that introns 17 and 30 are joined together via AAAT insertion (FIG. 2A/3A). To identify the most suitable sgRNAs for this experiment, all intronic regions were analyzed within the duplication for the computationally predicted most active guides. All guides with a predicted activity score greater than 0.75 were next analyzed using CRISPR Design Tool and ranked according to the least possible number of potential off-target sites. The 4 best guides (Table 1/3) were then subcloned into the lentiCRISPR 2 vector and one guide from the DMD guides was selected. Two duplication removal strategies were tested (FIG. 2B). Patient 1's fibroblasts were transduced with adeno-MyoD to induce transdifferentiation of fibroblasts into myoblasts. Simultaneously, they were transduced with lentiviral vector containing single (S6141; Strategy, single Dup18-30: sgRNA 1) or two (Junction and S275; Strategy 2) guide(s). The sgRNA was chosen as it was predicted to have the highest activity (25) and lowest possible off-target hits within the genome (28).

Three days later, puromycin was added to enrich for cells containing the lentiCRISPR 2-guide constructs and DNA was collected 7 days post puromycin selection. The cells were further differentiated towards myotubes and proteins were collected 7 days post-differentiation. The experiment was performed in triplicate. To determine if there was evidence of duplication removal on a molecular level a three-way PCR strategy outlined in FIG. 2C/3C was used. Briefly, Primers 1 and 3 (P1, P3) are universal to the region of interest, whereas Primer 2 (P2) is specific to the junction of the duplicated region. PCR amplification using combination of P1, P2, and P3 in patient DNA will results in two bands of 108 bp and 134 bp, which correspond to P1-P2- and P2-P3-derived amplicons, respectively. In contrast, only P1-P2-derived amplicon will be observed in healthy control (FIG. 2C/3C).

As seen in FIG. 2D/3D, the ratio of the two bands in the untreated control is 1:1. However the ratio becomes skewed towards the top band after lentiCRISPR treatment with guide S6141 (sgRNA 1) (Duplicated: Healthy =3:5) but not the Junction S275 or LentiGFP guides (Duplicated: Healthy=5.5:5) (FIG. 2E-F/3E, p<0.05). This indicates that there is a conversion of duplicated alleles to the healthy single copy. Furthermore, after analyzing dystrophin expression, full-length dystrophin in myotubes edited with guide S6141 similar to WT myotubes control was detected, but not in myotubes transduced with GFP or Junction+S275 guides+Cas9 cells(FIG. 2E-F/3E). So expression of full-length dystrophin (4.42%) in transdifferentiated myotubes treated with the single Dup18-30: sgRNA 1 was restored and is accompanied by restoration of a-dystroglycan, a critical component of the DGC (FIG. 3E).

The data demonstrate that CRISPR/Cas9-mediated removal of duplications leads to restoration of full-length protein expression in myotubes, which for the first time opens up entirely new treatment strategies for DMD patients with duplications.

### Example 3

### CRISPR/Cas9-Mediated Removal of MECP2 Duplication Syndrome

Using a similar approach it was demonstrated that the approach could be a therapeutic option for patients with genetic disorders other than DMD that also arise from duplication mutations, namely the MECP2 duplication syndrome. Methyl CpG Binding Protein 2 (MECP2) duplication syndrome, is a rare condition associated with intellectual disability and macrocephaly. It is caused by a variably sized CNV on the X-chromosome that includes a duplication of the MECP2 gene.

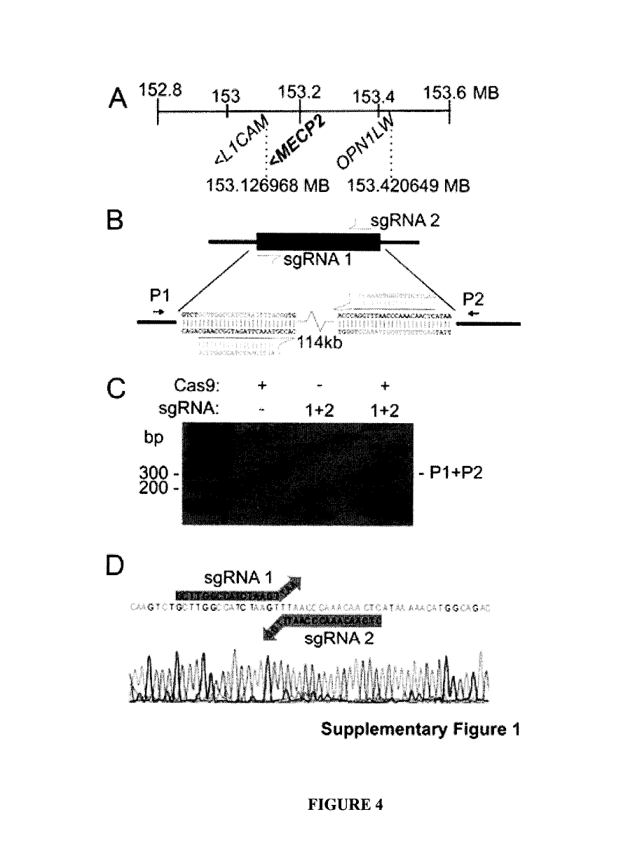

In order to explore whether the CRISPR/Cas9 technology could be utilized to target this CNV, it was first determined the exact orientation and breakpoint junction sequence of the duplication in a male patient with this disorder to be chrX:153,420,649-153,142,419 (hg19) duplication with nucleotides CA inserted at the breakpoint junction (FIG. 4A/6A).

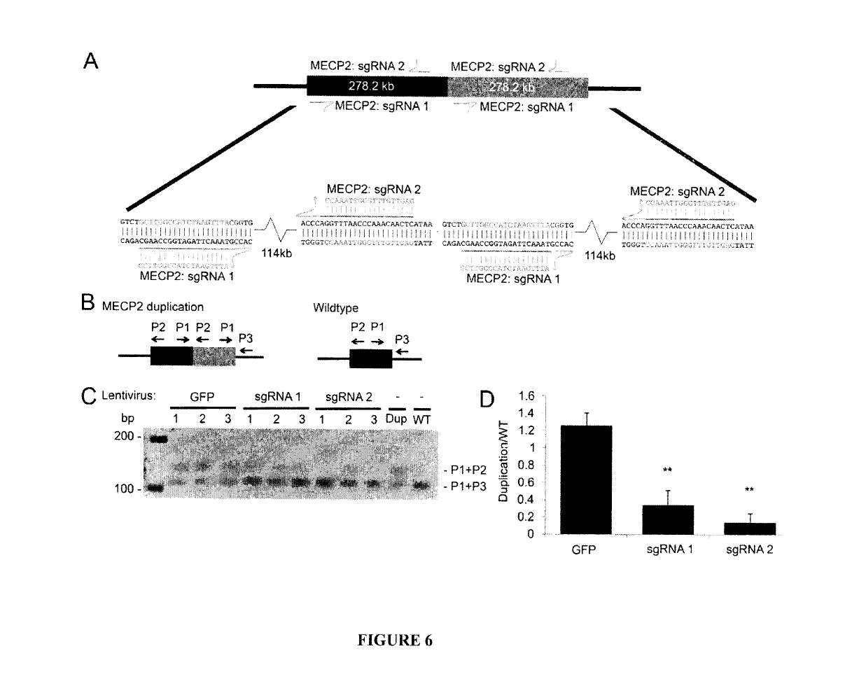

sgRNAs were designed and selected based on the fewest predicted off-target sites, designated as guides 75 and 80. These target regions outside of any known genes or regulatory elements in a 278 kb X-chromosomal duplication that included the MECP2 gene (Table 3). As a proof-of-concept, this 114 kb fragment was deleted by co-nucleofecting Cas9 nuclease plasmids (Addgene #48138) containing the sgRNA guides (FIGS. 4 and 6) into a control human cell line and used a three-way PCR-based assay to look for deletion products. Briefly, the intervening fragment between guide 75 and 80 can only be amplified if the 114 kb fragment was deleted. A band corresponding to a deletion only in cells nucleofected with the 2 sgRNA coupled with Cas9 was detected (FIG. 4C). The activity of these guides in primary fibroblasts of a healthy control using MECP2: sgRNAs 1 (g75) and 2 (g80) with SpCas9 were tested, and the deletion process of a 114 kb fragment was shown to occur through precise end joining of the two ends upon the two staggered Cas9-mediated double strand breaks (DSBs) (FIG. 4D).

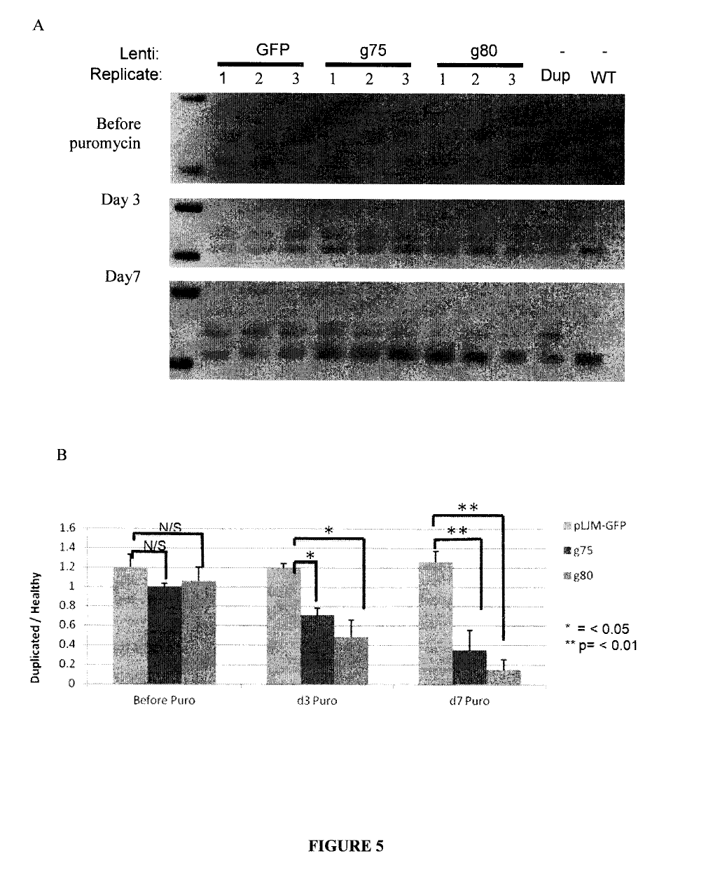

Knowing that the guides were active Strategy 1 was employed, as previously tested in DMD duplication removal, to primary dermal fibroblasts from a male patient with MECP2 duplication syndrome. These cells were transduced with lentiCRISPR containing guide 75 or guide 80 (sgRNA 1 or 2), and DNA was collected at various time points during puromycin selections (FIGS. 5 A and B was before and at days 3 and 7 post transduction and FIGS. 6 C and D was at 10 days post transduction). To test for duplication removal, similar molecular detection strategy as described in FIGS. 2C/3C using primers specific to the MECP2 locus was used (FIG. 6B), where Primers 1 and 3 (P1+P3) are universal to the region of interest and amplify the WT, single copy junction, whereas Primer 2 (P2) is specific to the junction of the duplicated region (FIG. 4B/6B). Once again, a loss of the amplicon specific to the duplicated region and an accumulation of the amplicon corresponding to a single WT copy was detected, indicating that the duplication was removed after treatment with lentiCRISPR (FIG. 5A-B/7C-D; p<0.01).

The results establish that a single sgRNA approach provides a novel, highly efficient therapeutic strategy to remove chromosomal duplications that can be explored for a number of different disorders caused by CNVs.

### Example 4

### Removal of Duplications in Vivo in Dmd Exons 18-30 Duplication Mouse Model

The following examples were conducted to test whether the duplication removal strategy is suitable for in vivo applications.

I. Generation of Dmd Exons 1840 Duplication (dup18-30 Dmd) Mouse Model

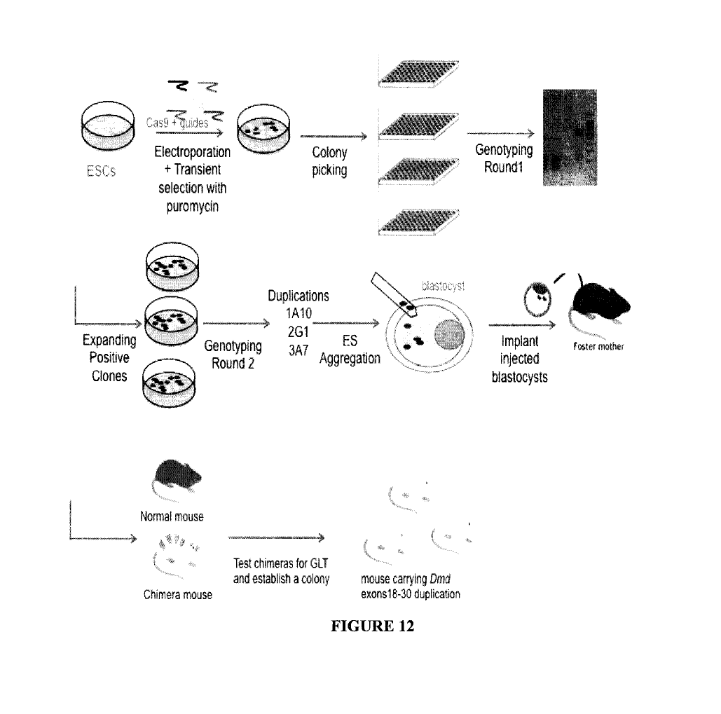

FIG. 12 illustrates the methodology used in generating the Dmd exons 18-30 duplication mouse model using a engineered targeted endonuclease technology.

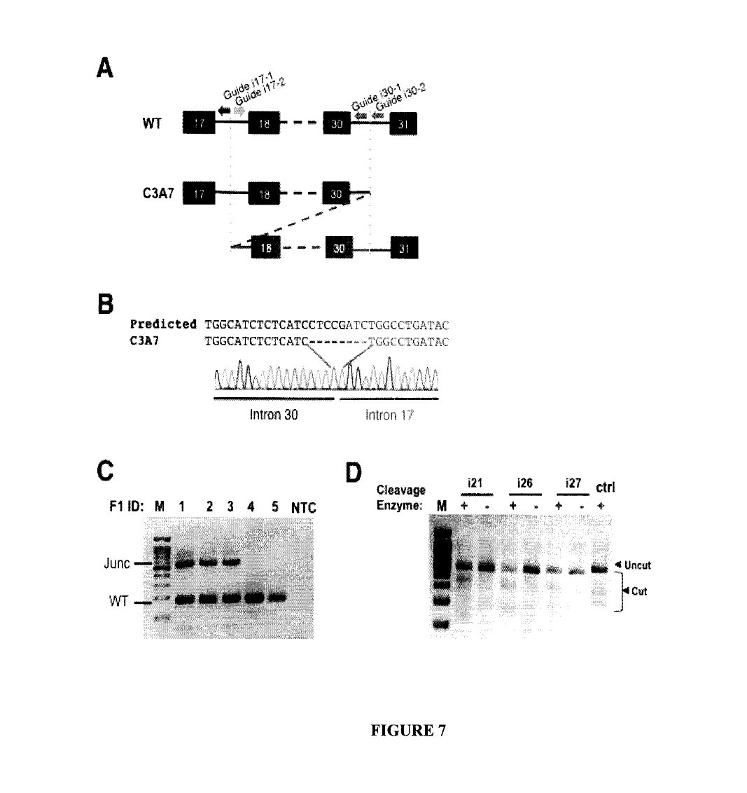

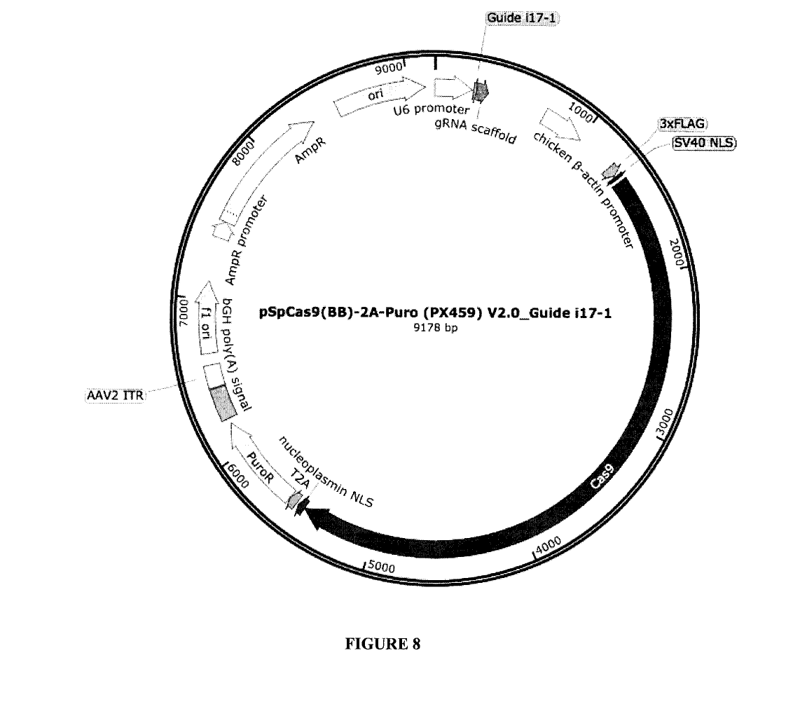

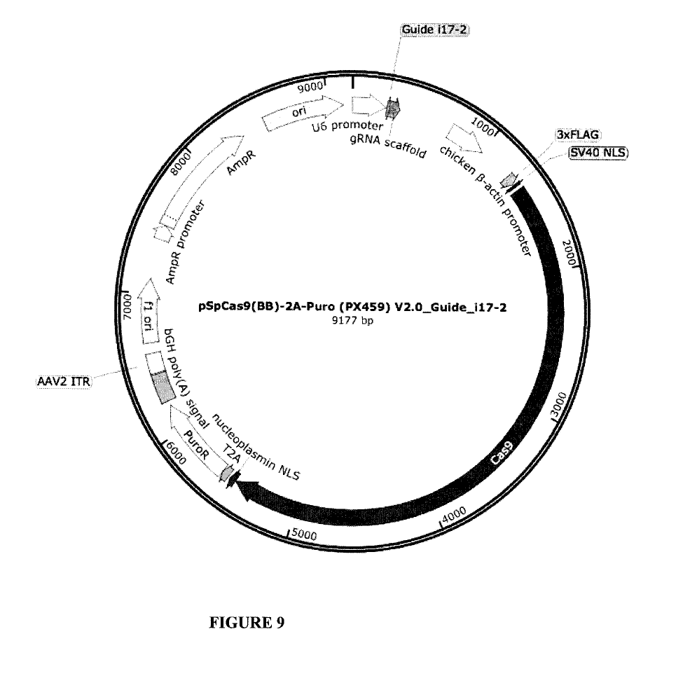

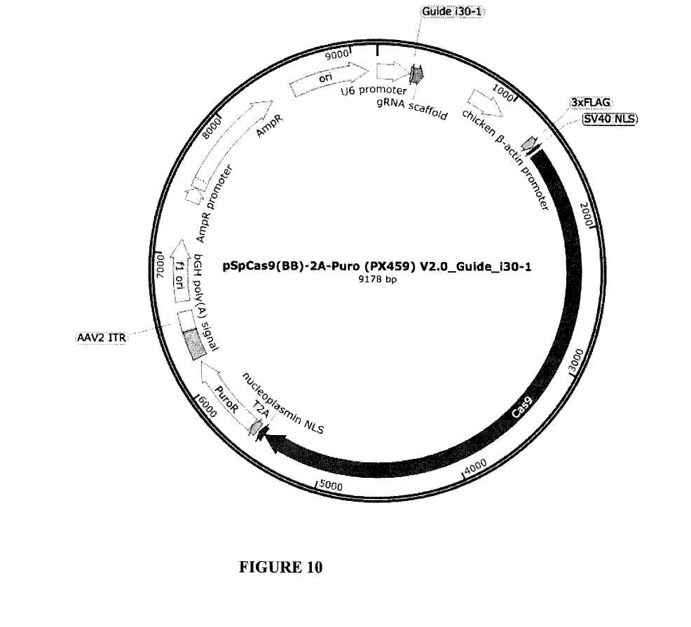

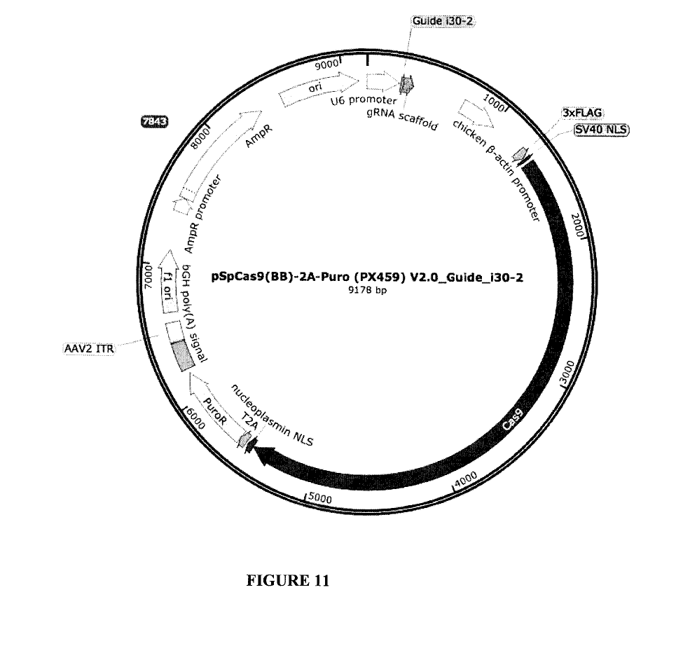

The dup18-30 Dmd mouse model was generated using 4 sgRNAs, 2 towards either end of the intended duplication, in intron 17 (i17-1 and i-17-2) and intron 30 (i30-1 and i-30-2) (FIG. 7A, Table 4). Individual guides were cloned into px459v2 plasmid (Addgene 62988) using standard techniques (49). The four plasmids were: pSPCas9(BB)-2A-Puro (PX459 V2.0_Guide i17-1 (FIG. 8; SEQ. ID. NO. 16); pSPCas9(BB)-2A-Puro (PX459 V2.0_Guide i17-2 (FIG. 9; SEQ. ID. NO. 17); pSPCas9(BB)-2A-Puro (PX459 V2.0_Guide i30-1 (FIG. 10; SEQ. ID. NO. 18); and pSPCas9(BB)-2A-Puro (PX459 V2.0_Guide 130-2 (FIG. 11; SEQ. ID. NO. 19). Plasmids were co-electroporated into 129XC57BL/6 F1 hybrid(G4)(62) derived mouse embryotic stem cells (mESCs) at the Toronto Center for Phenogenomics. Individual clones were generated based on Kraft et al(63). Briefly, successfully electroporated mESCs were selected using puromycin and the resulting clones were subjected for two-rounds of genotyping with primers P1 R 5′-GGAGAAGATCCAGAACCGTAGTG-3′ (SEQ. ID. NO. 12); P2 F 5′-CCTGTCCTGCTATTATTACTGGATG-3′ (SEQ. ID. NO. 13); and P3 R 5′-GCTAAACGTTGAGGCTACAGTTTG-3′ (SEQ. ID. NO. 14). Positive clones were identified and further expanded. One of the clones (3A7) containing the duplication junction, was then aggregated into blastocyst of a pregnant foster mouse. The resulting chimeras were crossed to establish a germline transmission of an exons18-30 duplication mouse model.

FIG. 7A is a schematic depicting duplication of exons 18-30 (red/shaded) and guides used to generate the dup 18-30 Dmd mouse model. FIG. 7B is a sequencing read of the duplication junction in mESCs clone 3A7 aligned to a predicted duplication sequence that is based on the prediction that a DSB will occur 3 bp before the PAM of a guide in intron 17 and 30. FIG. 7C is a PCR of tail-tissue using 3 primer PCR strategy that amplifies the duplication junction (Junc) and non duplicated sequence (WT). Of the 5 offspring of one 3A7 chimera, C1-3 F1 females but not C4-5 F1 males, showed germline transmission of this X-linked duplication. The NTC sample is a no DNA template control. FIG. 7D is a mutation detection assay for three tested sgRNAs targeting intron 21, 26, 27. i26 was found to be the most active sgRNA. The positive control for this mutation detection assay is a PCR from cells transfected with a known active sgRNA. Negative controls are samples where the mutation detection enzyme was not added.

II. In Vivo sgRNA Design and Testing

The guides targeting the exons18-30 duplication were designed based on a comultative ranking of the least possible number of potential off-target sites as analyzed by the CRISPR Design tool (28) and predicted activity according to Doench et al. (64) The best predicted sgRNAs (Table 5) were then subcloned into the lentiCRISPR v.2 vector (Addgene 52961)(48).

To experimentally test for the most active guide Neuro-2A cells were transfected with each plasmid using Lipofectamine and DNA collected 3 days later. Cell lysis, genomic PCR, and detection of cleavage were conducted using a GeneArt Genomic Cleavage Detection Kit (Life Technologies) according to the manufacturer's instructions. Briefly, 50 μl of cell lysis buffer and 2 μl of protein degrader were added to cell pellets and lysed in a thermal cycler (68° C. for 15 min and 95° C. for 10 min), genomic PCR was carried out using 1 μl of cell lysates and the following primers: for screening sgRNA i21 primers i21F 5′-AGGATTGCAGATTGCTTCAG-3′ (SEQ. ID. NO. 27) and i21R 5′-GGTGGAGAGAAACCAGATGC-3′ (SEQ. ID. NO. 28) were used, sgRNA i26: i26 F 5′-CATTTCACTGCTCTAGTTTTAATCCTG-3′(SEQ. ID. NO. 29) and i26R 5′-AAACACGCTTGAACTCAAATGCTAC-3′(SEQ. ID. NO. 30), sgRNA i27F: 5′-AGTGAGGTGCTCTATGGGAAATG-3′(SEQ. ID. NO. 32) and i27R 5′-CCATTTAAGAGTGGGTAACCAAGG-3′(SEQ. ID. NO. 31). The PCR products were subjected to re-annealing and a cleavage assay according to the manufacturer's instructions and then analyzed by electrophoresis in 2% agarose gels and ethidium bromide staining. Guide i26 was chosen for in vivo experimentation as it showed the highest degree of cleavage activity.

III. Lentiviral Vector Production

10 cm petri dish of 293T cells (ATCC) at 80% confluency were transfected with 10 μg of transfer LentiCRISPR plasmid containing GFP or sgRNA i26; 5 μg of the envelope (pCMV-VSV-G) (Addgene #8454) plasmid; and 7.5 pg of packaging (psPAX2) (Addgene 12260) plasmid using Calcium Phosphate transfection method. 60 hours post transfection, supernatant was collected, centrifuged at 3000 rpm for 10 minutes and filtered through 0.45 um low-binding filter (Whatman). Viral particles were aliquoted and stored in −80° C. until further use. The LentiCRISPR plasmid also comprisesSpCas9, as such the virus comprises both i26 gRNA and SpCas9 coding regions (SEQ. ID. NO. 25)

IV. Viral Injection

3 months old dup18-30 Dmd mice were anesthetized with isofluorane and injected via intramuscular route with 40 μl of viral particles into the right tibialis anterior (TA). The injection was repeated three days later, and the mice were euthanized 7 days after the last injection. Non-injected, contralateral TA muscles serve as controls.

V. Tissue Isolation and Cryosectioning

TA muscles were isolated and placed on cryomold in an upward orientation. The tissues were then snap frozen in liquid nitrogen-chilled isopentane for 2 minutes, wrapped in tin foil and stored at −80° C. freezer until further use. 8 μm thin cryosections were prepared from three different areas representing proximal, medial and distal (relative to the tendon) areas of the muscles. The slides were stored in −80° C. until further use.

VI. Immunofluorescence Staining for Dystrophin

Cryosectioned muscles were fixed in ice-cold methanol for 10 minutes and blocked with goat blocking buffer containing 3% BSA, 1% normal goat serum, and 0.3% Triton X-100 for 1 hour. The slides were then stained with undiluted mouse monoclonal antibody against dystrophin C-terminus (Novocastra, NCL-DYS2) for 1 hour at room temperature. Alexa 488-conjugated goat-anti mouse antibody (Thermo Fisher) was used as secondary reagent at a concentration of 1:500 for 30 minutes and nuclei were counter-stained with DAPI (Thermo Fisher) at a concentration of 2 ng/μl for 15 minutes. The slides were sealed with coverslips and Prolong Gold mounting media (Thermo Fisher). Images were taken using Zeiss Epifluorescence inverted microscope and analyzed using Volocity software.

VII. Results

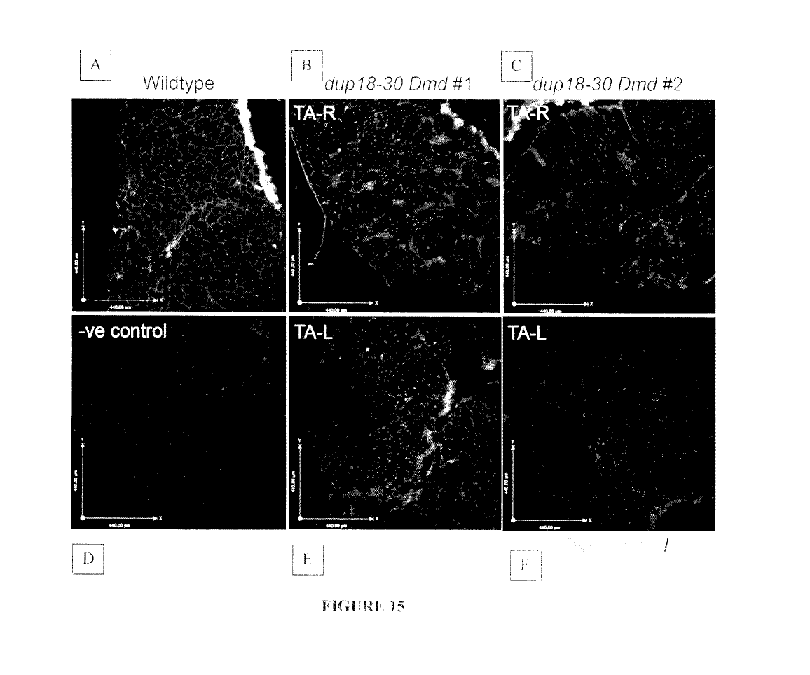

After tissue collection, cross sections of the right TA muscle (injected) and left TA muscle (non-injected) were stained with dystrophin specific antibodies. Dystrophin immunoreactivity was only detected in the right TA muscles indicating that the duplication removal strategy is successful and leads to the re-expression of dystrophin protein in treated muscles (FIG. 15).

More particularly, FIG. 15 illustrates detection of dystrophin by immunofluorescence staining after duplication removal in vivo.

8 μm-cross section of tibialis anterior (TA) muscle isolated from wildtype (C57BI/6J) and Dmd dup18-30 mice were stained for expression of dystrophin and counterstained with DAPI to identify the nuclei. Expression of dystrophin is detected in the right TA (TA-R) of Dmd dup18-30 mice (FIGS. 15 B and C), injected with lentiviral vector carrying i26 guides, but is absent in the non-injected, contralateral TA (TA-L) (FIGS. 15 E and F). TA muscle stained with secondary antibody alone served as a negative control (FIG. 15 D).

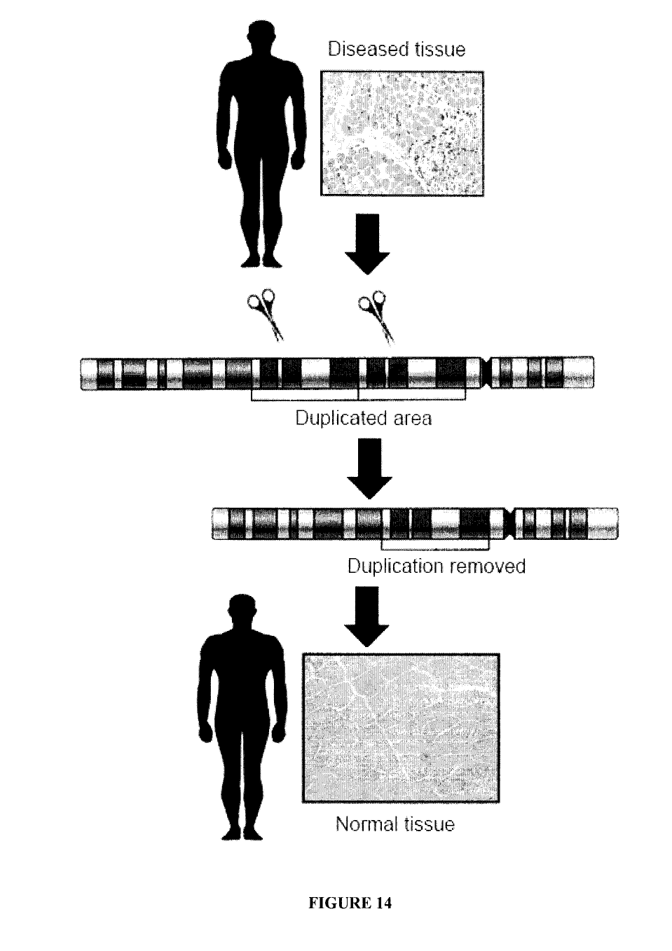

As such, the in vivo tests show that the methods of the invention can be used in vivo as illustrated in FIG. 14, where the method of the present invention can be used to treat a disease caused by a duplicative (or replicative) genetic material by removing the duplication, resulting in one copy.

TABLE 1
sgRNA Target/Coding DNA sequences and identities

| | | | | # OFF- | # OFF- | DESIGN |
|---|---|---|---|---|---|---|
| | sgRNA | GUIDE TARGET/CODING | | TARGETS | TARGETS | TOOL |
| GENE | ID | SEQUENCE* | PAM | INTRONIC | EXONIC | SCORE³⁷ |
| DMD | S6141 | ATATCTTCTTAAATACCCGA (SEQ. | GGG | 229 | 9 | 75 |
| | | ID. NO. 7) | | | | |
| DMD | S275 | AGTCTCACAAAACTGCAGAG (SEQ. | CGG | 10 | 0 | 98 |
| | | ID. NO. 8) | | | | |
| DMD | junc | TACTTATGTATTTTAAAAAC (SEQ. | CGG | 501 | 28 | 18 |
| | | ID. NO. 9) | | | | |
| X-Chromosome | Guide 75 | GCTTGGCCATCTAAGTTTA (SEQ. | CGG | 144 | 3 | 80 |
| MECP2 | | ID, NO. 10) | | | | |
| X-Chromosome | Guide 80 | GAGTTGTTTGGGTTAAACC (SEQ. | TGG | 144 | 3 | 80 |
| MECP2 | | ID. NO. 11) | | | | |

*the sgRNA target sequences would be the same as their coding sequence to produce the sgRNA. The sgRNA would be the same sequence as the noted except that it would be RNA where T (thymine) is replaced with U (uracil) (See. SEQ. ID. Nos 40-44). Variations between the target and sgRNA or its coding sequence may be acceptable as long as the resulting sgRNA is sufficiently complementary to the target sequence to bind the target sequence and direct the endonuclease to the cleavage site.

TABLE 2
Primers used for three way PCR assays

| | PRIMER NAME | SEQUENCE 5′-3′ |
|---|---|---|
| P1 | dup 18-30 common f | cag cat cat gac ctg ttt caa tc (SEQ. |
| | | ID. NO. 1) |
| P2 | dup 18-30 junc r | ttg tta gag ggc agc aag ttt gt (SEQ. |
| | | ID. NO. 2) |
| P3 | dup18-30 bound r | aag aag gct gtc acc agg aac t (SEQ. |
| | | ID. NO. 3) |
| P1 | mecp2 dup common | ccc aca gag tag agt gga gca g (SEQ. |
| | | ID. NO. 4) |
| P2 | mecp2 dup | cca cag cct atg aaa agg aca ag (SEQ. |
| | | ID. NO. 5) |
| P3 | mecp2dupout | tta gac aga gtc tca ctc cat cac |
| | | c (SEQ. ID. NO. 6) |

TABLE 3
Extended Table with off-target analysis.
Scores are calculated based on previously reported algorithm²³. Locus position is
determined and aligned to hg19. N/D represents non-detected indel percentage.
MECP2: sgRNA 1

| | Target DNA Sequence with their | | Locus | |
|---|---|---|---|---|
| Targets | corresponding PAM Sequence | Score | (Gene ID)* | Indels (%) |
| ON | GCTTGGCCATCTAAGTTTACGG | 100 | chrX:+153273069 | N/D |
| OFF 1 | AGGTTGCCCATCTAAGTTTATAG | 1.7 | chr8:+119018322 | N/D |
| OFF 2 | AAGTTGGCCTTCTAAGTTTAAAG | 1.3 | chr18:-70219106 | N/D |
| OFF 3 | TGAATGTTCATCTAAGTTTAGAG | 0.9 | chr12:-55085547 | N/D |
| OFF 4 | TTCTTGAGCGTCTAAGTTTAAGG | 0.9 | chr11:-125813383 | N/D |
| OFF 5 | TGGTAGCCCCTCTAAGTTTAAGG | 0.9 | chr9:-73214989 | N/D |
| OFF 6 | TGCTTGACCTCCTAAGTTTAAAG | 0.8 | chrX:-139329098 | N/D |
| OFF 7 | CATTTTGCCATCTAAGTTTATGG | 0.8 | chr5:-53752882 | 0.86 |
| OFF 8 | TACACTGCCATCTAAGTTTAGAG | 0.8 | chr17:+35634370 | N/D |
| OFF 9 | TTCTTGGAAGTCTAAGTTTAAGG | 0.8 | chr6:-91201638 | N/D |
| OFF 10 | TGATGTGCCTTCTAAGTTTAAAG | 0.8 | chrX:-43157660 | N/D |
| OFF 11 | AACATGGCCATCTAAGTTTTGGG | 0.7 | chr4:-160123587 | N/D |
| OFF 12 | TCGTTGGACATTTAAGTTTAGAG | 0.7 | chr12:+66851859 | N/D |
| OFF 13 | AGCTGGGCCTTCTAAGTTTGAAG | 0.7 | chr7:-16277369 | N/D |
| OFF 14 | TTCTTGGACTTCTAAGTTTGTGG | 0.6 | chr7:-129847577 | N/D |
| OFF 15 | TTCATGGACATCCAAGTTTATAG | 0.6 | chr1:+72535577 | N/D |
| OFF 16 | TGTTTGGCCCTCTAAATTTAAGG | 0.5 | chr14:-45589728 | N/D |
| OFF 17 | TGTTTGGCCTTCTAATTTTAAGG | 0.5 | chr2:+155525161 | N/D |
| OFF 18 | TTCTAGCCCATCTAAGTTTTCAG | 0.5 | chr3:-6566451 | N/D |
| OFF 19 | TGCAAGCCCATCTAAGTTTTAAG | 0.5 | chr5:-59096266 | N/D |
| OFF 20 | TTCTTGTCCCTCTAAGTTTCTGG | 0.4 | chr17:-75376272 | N/D |

MECP2: sgRNA 2

| | Target DNA Sequence with their | | Locus | |
|---|---|---|---|---|
| Targets | corresponding PAM Sequence | Score | (Gene ID)* | Indels (%) |
| ON | GAGTTGTTTGGGTTAAACCTGG | 100 | chrX:-153386812 | N/D |
| OFF 1 | TGTGTTGTTTGGGTGAAACCGAG | 2.7 | chr5:-20399210 | N/D |
| OFF 2 | TGAGTTTTTTGGGTTAAGCCTGG | 1.2 | chr7:+32206618 | N/D |
| OFF 3 | ACTGTTCTTTGGGTTAAACCAAG | 0.9 | chr3:-33704075 | N/D |
| OFF 4 | AGTTTTGTTTTGGTTAAACCTAG | 0.8 | chr16:-20201186 | N/D |
| OFF 5 | TGAGCTATTTGGTTTAAACCAAG | 0.7 | chr22:+26856195 | N/D |
| OFF 6 | GCAGATGTTTGGATTAAACCAAG | 0.6 | chr8:-65798479 | 0.87 |
| OFF 7 | GAAGATGTTTGGGTTAAAACTAG | 0.5 | chrX:-116193921 | N/D |
| OFF 8 | TGAATTCCTTTGGTTAAACCCAG | 0.5 | chr18:-73217777 | N/D |
| OFF 9 | TGAGGTCTCAGGGTTAAACCAAG | 0.5 | chr5:-2440574 | N/D |
| OFF 10 | TCATTTCTTTGGGTTAAACATAG | 0.5 | chr8:-22631876 | N/D |
| OFF 11 | TGAGATGATGGGGTTAAAGCTGG | 0.5 | chr17:+74157644 | N/D |
| | | | (NM_052916) | |
| OFF 12 | TTATTGGTTTGAGTTAAACCAAG | 0.4 | chr5:+115222903 | N/D |
| OFF 13 | AGGGTTTTTTGGCTTAAACCTAG | 0.4 | chr6:+151273474 | N/D |
| OFF 14 | TGGATTGTTTTTGTTAAACCTGG | 0.4 | chr2:-55249220 | N/D |
| OFF 15 | TGACTAGTTGGGGTTAAACATGG | 0.4 | chr20:+55793327 | N/D |
| OFF 16 | TGAGTTGAAAGGGTTAAACACAG | 0.4 | chr1:+180894617 | N/D |
| OFF 17 | TTTGTTCTTTGGGTTAAAGCCAG | 0.3 | chr18:+4272341 | N/D |
| OFF 18 | TGAGCCATATGGGTTAAACCAGG | 0.3 | chr5:-58907249 | 1.00 |
| OFF 19 | TTAGTCATTTCGGTTAAACCAGG | 0.3 | chr10:+15514525 | N/D |
| OFF 20 | AGAGGTGTGTGGGTTAAAGCAGG | 0.3 | chr4:+11194960 | N/D |

Dup18-30: sgRNA 1

| | Target DNA Sequence with their | | Locus | |
|---|---|---|---|---|
| Targets | corresponding PAM Sequence | Score | (Gene ID)* | Indels (%) |
| ON | ATATCTTCTTAAATACCCGAAGG | 100 | chrX:+32461612 | N/D |
| OFF 1 | AGTGTCTTCTTAAATACCTGCAG | 1.1 | chr2:+225344609 | N/D |
| OFF 2 | AAAACCTTCACAAATACCCGGAG | 0.7 | chr6:+19965267 | N/D |
| OFF 3 | AATCTCTTCTTCAATACCCTTGG | 0.7 | chr18:+23119458 | N/D |
| OFF 4 | AAGAGCTGCTTAAATACCCTGAG | 0.7 | chr11:+46836502 | 2.53 |
| OFF 5 | CATATCTTCTTAAATAGCCTTGG | 0.6 | chr8:+67600685 | N/D |
| OFF 6 | ATTAGCATCTTTAATACCCGAAG | 0.5 | chr7:+130907163 | N/D |
| OFF 7 | GATATATCCTGAAATACCCGTAG | 0.5 | chrX:-7222056 | N/D |
| OFF 8 | ATTTTCTTATGAAATACCCGAAG | 0.5 | chr14:-91038783 | N/D |
| OFF 9 | TAAATCCTCTTAAATACCCTAAG | 0.5 | chr1:-19924480 | N/D |
| OFF 10 | GAAATCTTCATAAATACCAGGAG | 0.5 | chr9:-34930580 | N/D |
| OFF 11 | AATTACTTCATAAATACCTGAGG | 0.5 | chr5:-170343578 | N/D |
| | | | (NM_022897) | |
| OFF 12 | AATTTCAACTTAAATACCCTTGG | 0.5 | chr13:-109558634 | 1.26 |
| OFF 13 | AATTCCATCTTAAATACCCTAAG | 0.5 | chr7:-10351725 | N/D |
| OFF 14 | CATCTTTTCTTAAATACCCAAGG | 0.4 | chr2:+208081568 | N/D |
| OFF 15 | AGTTTCTTGTTAAATACCCAAGG | 0.4 | chr6:+8264834 | N/D |
| OFF 16 | ATTCTCTTTTTAAATACCCACAG | 0.4 | chr5:+146530145 | N/D |
| OFF 17 | AATTTCTCTTTAAATACCCAAAG | 0.4 | chr19:-22631201 | N/D |
| OFF 18 | AATATTTTCTTCAATACCCCTGG | 0.4 | chr5:-146140359 | N/D |
| OFF 19 | AATATTTTCTTCAATACCCTAAG | 0.4 | chrl :-185743949 | N/D |
| OFF 20 | AATACCTTCATAAGTACCCGAAG | 0.4 | chr13:+23073576 | N/D |

TABLE 4
List of sgRNAs Targets/Coding Sequence and their corresponding PAMs
of Example 5.

| Oligo | | | | |
|---|---|---|---|---|
| Name | Targeting | Target/Coding Sequence 5′-3′ | PAM | Purpose |
| Guide | Intron 17 | AATACTACTAGCTCACCATC (SEQ. | TGG | Generating |
| i17-1 | | ID. NO. 15) | | duplication |
| | | | | 18-30 mouse |
| Guide | Intron 17 | GCATGGCGCAAAGGTCAAGA (SEQ. | AGG | Generating |
| i17-2 | | ID. NO. 17) | | duplication |
| | | | | 18-30 mouse |
| Guide | Intron 30 | ACTGGTGAAATCGTGCCCGG (SEQ. | AGG | Generating |
| i30-1 | | ID. NO. 19) | | duplication |
| | | | | 18-30 mouse |
| Guide | Intron 30 | CTTAGAAACATTCGTCAAGG (SEQ. | AGG | Generating |
| i30-2 | | ID. NO. 21) | | duplication |
| | | | | 18-30 mouse |
| Guide | Intron 21 | GTGGGATGGATCCCCGGATA (SEQ. | TGG | Duplication |
| i21 | | ID. NO. 23) | | removal |
| Guide | intron 26 | TAGCATTTGAGTTCAAGCGT (SEQ. | TGG | Duplication |
| i26 | | ID. NO. 24) | | removal |
| Guide | Intron 27 | CCCGGTACAGGGGAACGCCA (SEQ. | GGG | Duplication |
| i27 | | ID. NO. 26) | | removal |

*the sgRNA target sequences would be the same as their coding sequence to produce the sgRNA. The sgRNA would be the same sequence as the noted except that it would be RNA where T (thymine) is replaced with U (uracil) (See SEQ. ID. Nos. 33-39). Variations between the target and sgRNA or its coding sequence may be acceptable as long as the resulting sgRNA is sufficiently complementary to the target sequence to bind the target sequence and direct the endonuclease to the cleavage site.

### REFERENCES

1. R. Barrangou et al., CRISPR provides acquired resistance against viruses in prokaryotes. Science 315, 1709 (2007).
2. F. J. Mojica, C. Diez-Villasenor, J. Garcia-Martinez, E. Soria, Intervening sequences of regularly spaced prokaryotic repeats derive from foreign genetic elements. Journal of molecular evolution 60, 174 (2005).
3. C. Pourcel, G. Salvignol, G. Vergnaud, CRISPR elements in Yersinia pestis acquire new repeats by preferential uptake of bacteriophage DNA, and provide additional tools for evolutionary studies. Microbiology 151, 653 (2005).
4. A. Bolotin, B. Quinquis, A. Sorokin, S. D. Ehrlich, Clustered regularly interspaced short palindrome repeats (CRISPRs) have spacers of extrachromosomal origin. Microbiology 151, 2551 (2005).
5. B. Wiedenheft, S. S., J. A. Doudna, RNA-guided genetic silencing systems in bacteria and archaea. Nature 482, 331 (2012).
6. L. Cong et al., Multiplex genome engineering using CRISPR/Cas systems. Science 339, 819 (2013).
7. P. Mali et al., RNA-guided human genome engineering via Cas9. Science 339, 823 (2013).
8. E. Deltcheva et al., CRISPR RNA maturation by trans-encoded small RNA and host factor RNase III. Nature 471, 602 (2011).
9. M. Jinek et al., A programmable dual-RNA-guided DNA endonuclease in adaptive bacterial immunity. Science 337, 816 (2012).
10. Y. Fu, J. D. Sander, D. Reyon, V. M. Cascio, J. K. Joung, Improving CRISPR-Cas nuclease specificity using truncated guide RNAs. Nature biotechnology 32, 279 (2014).
11. F. J. Mojica, C. Diez-Villasenor, J. Garcia-Martinez, C. Almendros, Short motif sequences determine the targets of the prokaryotic CRISPR defence system. Microbiology 155, 733 (2009).
12. P. D. Hsu, E. S. Lander, F. Zhang, Development and applications of CRISPR-Cas9 for genome engineering. Cell 157, 1262 (2014).
13. L. S. Qi et al., Repurposing CRISPR as an RNA-guided platform for sequence-specific control of gene expression. Cell 152, 1173 (2013).
14. L. A. Gilbert et al., CRISPR-mediated modular RNA-guided regulation of transcription in eukaryotes. Cell 154, 442 (2013).
15. P. Perez-Pinera et al., Synergistic and tunable human gene activation by combinations of synthetic transcription factors. Nature methods 10, 239 (2013).
16. P. Mali et al., CAS9 transcriptional activators for target specificity screening and paired nickases for cooperative genome engineering. Nature biotechnology 31, 833 (2013).
17. M. L. Maeder et al., CRISPR RNA-guided activation of endogenous human genes. Nature methods 10, 977 (2013).
18. G. Schwank et al., Functional repair of CFTR by CRISPR/Cas9 in intestinal stem cell organoids of cystic fibrosis patients. Cell Stem Cell 13, 653 (2013).
19. X. Huang et al., Production of Gene-Corrected Adult Beta Globin Protein in Human Erythrocytes Differentiated from Patient iPSCs After Genome Editing of the Sickle Point Mutation. Stem cells 33, 1470 (2015).
20. D. G. Ousterout et al., Multiplex CRISPR/Cas9-based genome editing for correction of dystrophin mutations that cause Duchenne muscular dystrophy. Nature communications 6, 6244 (2015).
21. H. L. Li et al., Precise correction of the dystrophin gene in duchenne muscular dystrophy patient induced pluripotent stem cells by TALEN and CRISPR-Cas9. Stem cell reports 4, 143 (2015).
22. C. Long et al., Prevention of muscular dystrophy in mice by CRISPR/Cas9-mediated editing of germline DNA. Science 345, 1184 (2014).
23. T. S. Khurana, K. E. Davies, Pharmacological strategies for muscular dystrophy. Nature reviews. Drug discovery 2, 379 (2003).
24. A. W. Cheng et al., Multiplexed activation of endogenous genes by CRISPR-on, an RNA-guided transcriptional activator system. Cell research 23, 1163 (2013).
25. J. G. Doench et al., Rational design of highly active sgRNAs for CRISPR-Cas9-mediated gene inactivation. Nature biotechnology 32, 1262 (2014).
26. R. Redon et al., Global variation in copy number in the human genome. Nature 444, 444 (Nov. 23, 2006).
27. S. Tuffery-Giraud et al., Genotype-phenotype analysis in 2,405 patients with a dystrophinopathy using the UMD-DMD database: a model of nationwide knowledgebase. Human mutation 30, 934 (Jun, 2009).
28. P. D. Hsu et al., DNA targeting specificity of RNA-guided Cas9 nucleases. Nature biotechnology 31, 827 (2013).
29. R. J. Fairclough, M. J. Wood, K. E. Davies, Therapy for Duchenne muscular dystrophy: renewed optimism from genetic approaches. Nature reviews. Genetics 14, 373 (2013).
30. K. Bushby, F. Muntoni, A. Urtizberea, R. Hughes, R. Griggs, Report on the 124th ENMC International Workshop. Treatment of Duchenne muscular dystrophy; defining the gold standards of management in the use of corticosteroids. 2-4 Apr. 2004, Naarden, The Netherlands. Neuromuscular disorders : NMD 14, 526 (2004).
31. J. Tinsley et al., Expression of full-length utrophin prevents muscular dystrophy in mdx mice. Nature medicine 4, 1441 (1998).
32. P. Miura, B. J. Jasmin, Utrophin upregulation for treating Duchenne or Becker muscular dystrophy: how close are we? Trends in molecular medicine 12, 122 (2006).
33. J. M. Tinsley et al., Daily treatment with SMTC1100, a novel small molecule utrophin upregulator, dramatically reduces the dystrophic symptoms in the mdx mouse. PloS one 6, e19189 (2011).
34. A. P. Weir, E. A. Burton, G. Harrod, K. E. Davies, A- and B-utrophin have different expression patterns and are differentially up-regulated in mdx muscle. The Journal of biological chemistry 277, 45285 (2002).
35. R. J. Fairclough, K. J. Perkins, K. E. Davies, Pharmacologically targeting the primary defect and downstream pathology in Duchenne muscular dystrophy. Current gene therapy 12, 206 (2012).
36. E. Kimura, S. Li, P. Gregorevic, B. M. Fall, J. S. Chamberlain, Dystrophin delivery to muscles of mdx mice using lentiviral vectors leads to myogenic progenitor targeting and stable gene expression. Molecular therapy : the journal of the American Society of Gene Therapy 18, 206 (2010).
37. Y. Zhang, D. Duan, Novel mini-dystrophin gene dual adeno-associated virus vectors restore neuronal nitric oxide synthase expression at the sarcolemma. Human gene therapy 23, 98 (2012).
38. S. Cirak et al., Exon skipping and dystrophin restoration in patients with Duchenne muscular dystrophy after systemic phosphorodiamidate morpholino oligomer treatment: an open-label, phase 2, dose-escalation study. Lancet 378, 595 (2011).
39. N. M. Goemans et al., Systemic administration of PRO051 in Duchenne's muscular dystrophy. The New England journal of medicine 364, 1513 (2011).
40. Y. Aoki et al., Bodywide skipping of exons 45-55 in dystrophic mdx52 mice by systemic antisense delivery. Proceedings of the National Academy of Sciences of the United States of America 109, 13763 (2012).
41. S. Jarmin, H. Kymalainen, L. Popplewell, G. Dickson, New developments in the use of gene therapy to treat Duchenne muscular dystrophy. Expert opinion on biological therapy 14, 209 (Feb, 2014).
42. L. V. Nicholson et al., Integrated study of 100 patients with Xp21 linked muscular dystrophy using clinical, genetic, immunochemical, and histopathological data. Part 1. Trends across the clinical groups. Journal of medical genetics 30, 728 (1993).
43. L. V. Nicholson, K. M. Bushby, M. A. Johnson, D. Gardner-Medwin, I. B. Ginjaar, Dystrophin expression in Duchenne patients with “in-frame” gene deletions. Neuropediatrics 24, 93 (1993).
44. M. van Putten et al., Low dystrophin levels in heart can delay heart failure in mdx mice. Journal of molecular and cellular cardiology 69, 17 (2014).
45. M. van Putten et al., Low dystrophin levels increase survival and improve muscle pathology and function in dystrophin/utrophin double-knockout mice. FASEB journal:official publication of the Federation of American Societies for Experimental Biology 27, 2484 (2013).
46. M. van Putten et al., The effects of low levels of dystrophin on mouse muscle function and pathology. PloS one 7, e31937 (2012).
47. R. B. de Vrueh, E.R.F. de Haan, J.M.H. , “Update on 2004 Background Paper, BP 6.19 Rare Diseases” (2013).
48. N. E. Sanjana, O. Shalem, F. Zhang, Improved vectors and genome-wide libraries for CRISPR screening. Nature methods 11, 783 (2014).
49. F. A. Ran et al., Genome engineering using the CRISPR-Cas9 system. Nature protocols 8, 2281 (2013).
50. Sander, J. D. & Joung, J. K. CRISPR-Cas systems for editing, regulating and targeting genomes. Nature biotechnology 32, 347-355, doi:10.1038/nbt.2842 (2014).
51. Hsu, P. D., Lander, E. S. & Zhang, F. Development and applications of CRISPR-Cas9 for genome engineering. Cell 157, 1262-1278, doi:10.1016/j.cell.2014.05.010 (2014).
52. Garneau, J. E. et al. The CRISPR/Cas bacterial immune system cleaves bacteriophage and plasmid DNA. Nature 468, 67-71, doi:10.1038/nature09523 (2010).
53. Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586, doi:10.1073/pnas.1208507109 (2012).
54. Jinek, M. et al. A programmable dual-RNA-guided DNA endonuclease in adaptive bacterial immunity. Science 337, 816-821 (2012).
55. Mojica, F. J., Diez-Villasenor, C., Garcia-Martinez, J. & Almendros, C. Short motif sequences determine the targets of the prokaryotic CRISPR defence system. Microbiology 155, 733-740, doi:10.1099/mic.0.023960-0 (2009).
56. Barrangou, R. & Marraffini, L. A. CRISPR-Cas systems: Prokaryotes upgrade to adaptive immunity. Molecular cell 54, 234-244, doi:10.1016/j.molcel.2014.03.011 (2014).
57. Jansen, R., van Embden, J. D., Gaastra, W. & Schouls, L. M. Identification of a novel family of sequence repeats among prokaryotes. Omics : a journal of integrative biology 6, 23-33 (2002).
58. Sashital, D. G., Wiedenheft, B. & Doudna, J. A. Mechanism of foreign DNA selection in a bacterial adaptive immune system. Molecular cell 46, 606-615, doi:10.1016/j.molcel.2012.03.020 (2012).
59. Polakoff, R. J., Morton, A. A., Koch, K. D. & Rios, C. M. The psychosocial and cognitive impact of Duchenne's muscular dystrophy. Seminars in pediatric neurology 5, 116-123 (1998).
60. Sander, J. D. & Joung, J. K. CRISPR-Cas systems for editing, regulating and targeting genomes. Nature biotechnology 32, 347-355, doi:10.1038/nbt.2842 (2014).
61. Grieger, J. C., Choi, V. W. & Samulski, R. J. Production and characterization of adeno-associated viral vectors. Nature protocols 1, 1412-1428, doi:10.1038/nprot.2006.207 (2006).
62. George, S. H. et al. Developmental and adult phenotyping directly from mutant embryonic stem cells. Proceedings of the National Academy of Sciences of the United States of America 104, 4455-4460, doi:10.1073/pnas.0609277104 (2007).
63. Kraft, K. et al. Deletions, Inversions, Duplications: Engineering of Structural Variants using CRISPR/Cas in Mice. Cell Reports, doi:10.1016/j.celrep.2015.01.016 (2015).
64. Doench, J. G. et al. Rational design of highly active sgRNAs for CRISPR-Cas9-mediated gene inactivation. Nature Biotechnology 32, 1262-1267, doi:10.1038/nbt.3026 (2014).

What is claimed is:

1. A method of removing duplicate genetic material in vivo or in vitro in a eukaryotic cell wherein the duplication is a head to tail duplication, the method comprising: using one single guide RNA that can bind to a single target genomic DNA at each occurrence of said target on the genetic material and complex with an endonuclease, wherein the target is adjacent or sufficiently proximal to the endonuclease recognition site and wherein the guide and endonuclease complex forms a complex with the target and the endonuclease cleaves the target genetic material, thus removing the duplicate genetic material from the eukaryotic cell.

2. The method of claim 1 wherein the endonuclease is a Cas protein, Cas fusion protein, or Cas 9 protein.

3. The method of claim 1 wherein the method further comprises the step of repairing and joining the cleaved genetic material and restoring wild type gene expression in same.

4. The method of claim 1 for removing duplicate genetic material from a eukaryotic cell comprising:
(i) transfecting or transducing the eukaryotic cell with a nucleic acid encoding one single guide RNA complementary to genomic DNA within a duplicate region of the eukaryotic cell and a region that can interact and complex with an endonuclease Cas protein; and
(ii) transfecting or transducing the eukaryotic cell with a nucleic acid encoding an endonuclease Cas protein that interacts and can complex with the RNA and the target genetic material, wherein the eukaryotic cell expresses the guide RNA and the endonuclease Cas protein, the RNA binds to the complementary genomic DNA of the target genetic material and interacts and forms a complex with the endonuclease Cas protein, such that a complex forms at each copy of the target genetic material and removes the duplicate genetic material, restoring wild type genetic material.

5. The method of claim 1 wherein the eukaryitic cell is a human cell and the endonuclease is a human codon-optimized Cas9 protein.

6-8. (canceled)

9. The method of claim 3 wherein the human cell is selected from the group consisting of: oocytes, myoblasts, myocytes, and cardiomyocytes.

10-11. (canceled)

12. The method of claim 1 for treating a condition in a mammal that can benefit from deletion of head to tail duplicate genetic material, the method comprising administering to said mammal an effective amount of:
(i) a composition comprising a nucleic acid encoding one single guide RNA complementary to genomic DNA of the eukaryotic cell of said mammal and a region that can interact and complex with an endonuclease Cas protein; and
(ii) a composition comprising a nucleic acid encoding an endonuclease Cas protein that interacts and can complex with the RNA and the target genetic material;
wherein composition (i) and (ii) can optionally be one composition and wherein the nucleic acids of (i) and (ii) are incorporated into a vector and in a delivery vehicle suitable for delivering same to the eukaryotic cell of the mammal in a manner that enables the cell to be transfected with said nucleic acids and express same once transfected to remove the duplicate genetic material and restore single copy wild type of same in the cell.

13. The method of claim 12 wherein the condition in the mammal is Duchenne Muscular Dystrophy or MECP2 duplication syndrome.

14-16. (canceled)

17. A method for removing replicated genetic material present head to tail on a nucleotide sequence of a cell, such as a eukaryotic cell with or without an intervening sequence in-between the replicated genetic material, the method comprising:
(i) delivering to the cell one exogenous polynucleotide guide that binds to a target sequence at each copy of said target sequence within said replicate genetic material and wherein said target sequence at each occurrence has or is sufficiently proximal to an endonuclease recognition and cleavage site to enable an endonuclease complexed with the guide to complex with or bind the recognition site, wherein , delivery to the cell of the exogenous polynucleotide guide comprises delivering a nucleotide sequence encoding same to the cell for expression within the cell or delivering the guide per se to the cell;
(ii) delivering to the cell an endonuclease that can complex with the guide and recognizes the endonuclease recognition site, wherein delivering to the cell can include: delivering to the cell a nucleotide sequence encoding the endonuclease to the cell for expression within the cell; delivering to the cell the endonuclease per se or creating conditions suitable for expressing the endonuclease within the cell; and
(iii) the delivery of the guide and endonuclease is done in a manner so that they form an endonuclease complex where upon the guide binds to the target sequence, the endonuclease recognizes the endonuclease recognition site and creates a double stranded break at each cleavage site, thus removing the duplicate genetic material.

18. The method of claim 17, wherein the exogenous polynucleotide guide and endonuclease can be delivered to the cell by delivering their coding sequence to the cell on the same or different vectors.

19. The method of claim 18 wherein the vector is selected from plasmids, phages, cosmids, an RNA vector or protein.

20. The method of claim 17, wherein the guide and endonuclease can be delivered to the cell as a n endonuclease/guide complex.

21. The method of claim 20 wherein the delivery vehicle are selected from liposomes, electroporation.

22. The method of claim 17 further comprising the step of repairing and joining the cleaved nucleotide sequence.

23. The method of claim 22 wherein repair is done by non-homologous end joining.

24. The method of claim 23 wherein the one copy of the genetic material where before there was two or more.

25. The method of claim 17 wherein the replicate genetic material is present in duplicate or triplicate or more.

26. The method of claim 25 wherein the replicate genetic material is present in duplicate.

27. The method of claim 17 wherein the exogenous polynucleotide guide is delivered to the cell by:
(i) transfecting or transducing the cell with a vector comprising nucleic acid encoding the guide;
(ii) if the endonuclease is exogenous, transfecting the cell with a vector nucleic acid encoding the endonuclease;
wherein the cell expresses the guide and the endonuclease, the guide forming a complex with the endonuclease, and wherein the vector in (i) and (ii) can be the same or different.

28. The method of claim 26 wherein the duplicate genetic material is associated with a Duchenne's muscular dystrophy caused by duplicative genetic material.

29. The method of claim 28, wherein the duplication is a duplication of exons 18-30.

30. The method of claim 26, wherein the duplicate genetic material is associated with MECP2 duplication syndrome.

31. The method of claim 28 for prevention or treatment of said disease by delivering to the patient, or embryo or egg a guide and endonuclease or vector coding same or guide/endonuclease complex alone or together of the invention to remove the duplicate genetic material and restoring wild type function of the affected genetic material

32-35. (canceled)

36. A kit for the treatment of a condition caused by duplicate genetic material, selected from Duchenne's muscular dystrophy and MECP2 duplication syndrome comprising:
(a) One or more compositions comprising one or more of the following:
(i) a nucleic acid encoding one guide;
(ii) a nucleic acid encoding an endonuclease;
(iii) an endonuclease;
(iv) a guide;
(v) an endonuclease complexed with the guide RNA;
(vi) both iii and iv;
(vii) a vector comprising I and ii or two vectors comprising i and ii respectively; and
(viii) any one or more of the above and optionally a delivery vehicle selected from a bacterial plasmid, viral phage, cosmid, or a liposome; and optionally
(b) a pharmaceutically acceptable carrier; and
(c) optionally instructions for the treatment or prevention of a condition caused by duplicate genetic material, selected from Duchenne's muscular dystrophy and MECP2 duplication syndrome.

37-39. (canceled)

40. The method of claim 17 wherein the endonuclease is a Cas protein, a Cas fusion protein, or a Cas 9 protein.
